(19) United States
(12) Patent Application Publication
(10) Pub. No.: US 2003/0125338 A1
(43) Pub. Date: Jul. 3, 2003

(54) Compounds, compositions and methods for modulating beta-amyloid production

(75) Inventors: Bruce P. Connop, Vancouver (CA); Amelia Grant, Vancouver (CA); Parimal S. Nathwani, Burnaby (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: Active Pass Pharmaceuticals, Inc., Vancouver (CA)

(21) Appl. No.: 10/170,224

(22) Filed: Jun. 12, 2002

Related U.S. Application Data
(60) Provisional application No. 60/309,257, filed on Jul. 31, 2001.
(60) Provisional application No. 60/297,845, filed on Jun. 12, 2001.

(51) Int. Cl. A61K 31/4965; C 07D 4 3/02; C07D 241/02

(52) U.S. Cl. 514/255.06; 514/255.05; 544/405; 544/408

(57) ABSTRACT

Methods and compositions useful in the treatment of amyloidosis and conditions and diseases associated therewith, such as Alzheimer's disease, are provided. The methods involve administering to a subject in need thereof a pharmaceutical composition including one or more agents that modulate PPARα and/or PPARδ activity, resulting in an inhibition of β-amyloid production and/or release from cells of the subject, particularly brain cells.

### BACKGROUND OF THE INVENTION

1. Field of the Invention

The invention relates to compounds, compositions and methods for regulating the production and/or release of β-amyloid in cells, and provides for alleviation and prevention of amyloid production, release and/or plaque development.

2. Description of the Related Art

Alzheimer's disease (AD) is a common brain disorder of the elderly and is associated with progressive dementia. The key features of the disease include progressive memory impairment, loss of language and visuospatial skills, and behavior deficits. These changes in cognitive function are the result of degeneration of neurons in the cerebral cortex, hippocampus, basal forebrain, and other regions of the brain. Neuropathological analyses of postmortem Alzheimer's diseased brains consistently reveal the presence of large numbers of neurofibrillary tangles in degenerated neurons and neuritic plaques in the extracellular space and in the walls of the cerebral microvasculature. The neurofibrillary tangles are composed of bundles of paired helical filaments containing hyperphosphorylated tau protein (Lee, V. M and Trojanowski, J. Q. Curr. Opin. Neurobiol. 2:653, 1992). The neuritic plaques consist of deposits of proteinaceous material surrounding an amyloid core (Selkoe, D. J., Annu. Rev. Neurosci. 17:489-517, 1994).

Evidence suggests that deposition of amyloid-β peptide (Aβ) plays a significant role in the etiology of Alzheimer's disease. A portion of this evidence is based upon studies that have been generated from data with regard to familial Alzheimer's disease. To date, this aggressive form of Alzheimer's disease has been shown to be caused by missense mutations in (at least) three genes: the amyloid precursor protein (APP) gene itself (Goate, A. et al., Nature 349:704-706, 1991; Mullan, M. et al., Nature Genet. 1:345-347, 1992), and two genes termed presenilins 1 and 2 (Sherrington, R. et al., Nature 375:754-760, 1995; Rogaev, E. I. et al., Nature 376:775-778, 1995).

The missense mutations in APP are located in the region of the protein where proteolytic cleavage normally occurs, and expression of these mutants results in increased production of Aβ (Citron, M. et al., Nature 360:672-674, 1992, Cai, X-D. et al., Science 259:514-516 1993 and Reaume, A. G. et al., J Biol. Chem. 271:23380-23388, 1996). Analysis of over 75 mutations of the presenilin genes consistently reveals that these mutations which invariably lead to Alzheimer's disease also result in increased levels of the longer isoform of Aβ known as Aβ-42 (Scheuner, D. et al., Nature Medicine 2:864-870, 1996 and Selkoe, Physiological Reviews 81:741-766 (2001)). Thus, increased production of Aβ, and in particular Aβ-42 is associated with Alzheimer's disease.

Corroborating evidence has been derived from at least two other sources. First, transgenic mice that express either altered APP and/or presenilin genes exhibit neuritic plaques and age-dependent memory deficits (Games, D. et al., “Alzheimer-type neuropathology in transgenic mice overexpressing V717F β-amyloid precursor protein,” Nature 373:523-525 (1995); Masliah, E. et al., “Comparison of neurodegenerative pathology in transgenic mice overexpressing V717F β-amyloid precursor protein and Alzheimer's disease,” J Neurosci. 16:5795-5811 (1996); Hsiao, K. et al., “Correlative memory deficits, Aβ elevation, and amyloid plaques in transgenic mice,” Science 274:99-103 (1996); Holcomb et al., Nature Medicine 4:97-100 (1998)). The second piece of evidence comes from study of patients suffering from Down's syndrome, who develop amyloid plaques and other symptoms of Alzheimer's disease at an early age (Mann, D. M. A. and M. M. Esiri, “The pattern of acquisition of plaques and tangles in the brains of patients under 50 years of age with Down's syndrome,” J. Neurol. Sci. 89:169-179 (1989)). Because the APP gene is found on chromosome 21, it has been hypothesized that the increased gene dosage which results from the extra copy of this chromosome in Down's syndrome accounts for the early appearance of amyloid plaques (Kang, J. et al., “The precursor protein of Alzheimer's disease amyloid A4 protein resembles a cell-surface receptor,” Nature 325:733-736 (1987); Tanzi, R. E. et al., “Amyloid β protein gene: cDNA, mRNA distribution and genetic linkage near the Alzheimer locus,” Science 235:880-884 (1987)). Taken together with the evidence derived from cases of familial Alzheimer's disease, the current data suggest that genetic alterations that result in an increase in Aβ production can induce Alzheimer's disease. Accordingly, since Aβ deposition is an early and invariant event in Alzheimer's disease, it is believed that treatment that reduces production of Aβ will be useful in the treatment of this disease.

The principal component of the senile plaque is the 4 kDa β-amyloid peptide (Aβ). Ranging between 39 and 43 amino acids in length, Aβ is formed by endoproteolysis of APP. Alternative splicing generates several different isoforms of APP; in neurons, the predominant isoform is 695 amino acids in length (APP695). As APP traverses the endoplasmic reticulum (ER) and trans-Golgi network (TGN), it becomes N- and O-glycosylated and tyrosine-sulfated. Mature holoprotein can be catabolized in several compartments to produce both non- and amyloidogenic APP fragments.

APP is expressed and constitutively catabolized in most cells. The dominant catabolic pathway appears to be cleavage of APP within the Aβ sequence by an enzyme provisionally termed α-secretase, leading to release of a soluble ectodomain fragment known as APPsα. In contrast to this non-amyloidogenic pathway, APP can also be cleaved by enzymes known as β- and γ-secretase at the N- and C-termini of the Aβ, respectively, followed by release of Aβ into the extracellular space. To date, BACE has been identified as β-secretase (Vasser et al., Science 286:735-741, 1999) and presenilins have been implicated in γ-secretase activity (De Strooper et al., Nature 391:387-390, 1998)

The 39-43 amino acid Aβ peptide is produced by sequential proteolytic cleavage of the amyloid precursor protein (APP) by the enzyme(s) β and γ secretases. Although Aβ-40 is the predominant form produced, 5-7% of total Aβ exists as Aβ-42 (Cappai et al., Int. J. Biochem. Cell Biol. 31:885-889, 1999). The length of the Aβ peptide appears to dramatically alter its biochemical/biophysical properties. Specifically, the additional two amino acids at the C-terminus of Aβ-42 are very hydrophobic, presumably increasing the propensity of Aβ-42 to aggregate. For example, Jarrett et al. demonstrated that Aβ-42 aggregates very rapidly in vitro compared to Aβ-40, suggesting that the longer forms of Aβ may be the important pathological proteins that are involved in the initial seeding of the neuritic plaques in AD (Jarrett et al., Biochemistry 32:4693-4697, 1993; Jarrett et al., Ann. NY Acad. Sci. 695:144-148, 1993).

This hypothesis has been further substantiated by the recent analysis of the contributions of specific forms of Aβ in cases of genetic familial forms of AD (FAD). For example, the “London” mutant form of APP (APPV717I) linked to FAD selectively increases the production of Aβ42/43 forms versus Aβ40 (Suzuki et al., Science 264:1336-1340, 1994) while the “Swedish” mutant form of APP (APPK670N/M671L) increases levels of both Aβ-40 and Aβ-42/43 (Citron et al., Nature 360:672-674, 1992; Cai et al., Science 259:514-516, 1993). Also, it has been observed that FAD-linked mutations in the Presenilin-1 (PS1) or Presenilin-2 (PS2) genes will lead to a selective increase in Aβ-42/43 production but not Aβ-40 (Borchelt et al., Neuron 17:1005-1013, 1996). This finding was corroborated in transgenic mouse models expressing PS mutants that demonstrate a selective increase in brain Aβ-42 (Borchelt et al., Neuron 17:1005-1013, 1996; Duff et al., Neurodegeneration 5(4):293-298, 1996). Thus the leading hypothesis regarding the etiology of AD is that an increase in Aβ-42 production and/or release is a causative event in the disease pathology.

In addition to a relationship with coronary disease, a relationship exists between serum cholesterol levels and the incidence and the pathophysiology of AD. Epidemiological studies show that patients with elevated cholesterol levels have an increased risk of AD (Notkola et al., Neuroepidemiology. 17(1):14-20, 1998; Jarvik et al., Neurology. 45(6):1092-6, 1995). In addition to the data which suggests that elevated levels of Aβ are associated with AD, other environmental and genetic risk factors have been identified. The best studied of these is polymorphism of the apolipoprotein E (ApoE) gene: patients homozygous for the ε4 isoform of ApoE (apoE4) have consistently been shown to have an increased risk for AD (Strittmatter et al., Proc Natl Acad Sci USA 90:1977-1981 (1993). Because ApoE is a cholesterol transport protein, several groups have observed a correlation between the risk of developing AD and circulating levels of cholesterol (Mahley. Science. 240:622-630, 1998; Saunders et al., Neurology. 43:1467-1472, 1993; Corder et al., Science. 261:921-923, 1993; Jarvik et al., Annals of the New York Academy of Sciences. 826:128-146, 1997). Moreover, cholesterol loading increases the production of Aβ protein (Simons et al., PNAS. 95:6460-6464, 1998), while pharmacological reduction of cholesterol with the HMG CoA reductase inhibitor simvastatin decreases levels of both Aβ-40 and Aβ-42 (Fassbender et al., Proc Natl Acad Sci 98:5856-5861 (2001)) in vitro. Consistent with these data are the results of epidemiological studies which have shown that treatment with certain HMG CoA reductase inhibitors, commonly used to normalize cholesterol levels in humans, reduces the prevalence of AD (Wolozin et al., Arch Neurol 57:1439-1443 (2000); Jick et al., Lancet 356:1627-1631 (2000). Taken together, these data suggest a link between regulation of cholesterol levels and AD.

Collectively the wealth of data derived from 1) the biophysical properties of Aβ, 2) in vitro studies of various cell lines, 3) in vivo studies of transgenic mice and 4) analysis of humans with FAD mutations—all point to Aβ-42 as the key pathogenic protein in AD. Thus, there is a need for treatments which selectively inhibit the production and/or release of Aβ-42. Such treatments may prove to be extremely valuable in the treatment of both familial and/or sporadic cases of AD.

### BRIEF SUMMARY OF THE INVENTION

The invention provides a method for modulating the production and/or release of β-amyloid from a cell, comprising treating the cell with an agent, or a composition comprising an agent, that acts as a PPARα and/or PPARδ agonist; in one embodiment, the cell is a brain cell.

The invention further provides a method for modulating the production and/or release of β-amyloid from a cell using an agent selected from the group consisting of (2-pyrimidinylthio) alkanoic acids, esters, amides, hydrazides and 4- and 6-substituted derivatives thereof.

The invention still further provides a method of inhibiting extracellular amyloid levels in the brain of a human in need of such inhibition, comprising administering to the human a pharmaceutical composition comprising an agent that activates PPARα and/or PPARδ activity. In specific embodiments, the amyloid is β-amyloid-42.

In addition, the invention provides compounds, compositions and methods for regulating the production and/or release of β-amyloid in cells, and provides for alleviation and prevention of amyloid production, release and/or plaque development.

The invention yet further provides a method for preferentially reducing production and/or release of Aβ-42 relative to one or more other forms of Aβ, in a target that produces and/or releases Aβ-42, for instance a target selected from a cell, a human, a non-human mammal, and the brain of a human, comprising administering to the target a compound or pharmaceutical composition comprising a chemical agent as described herein. This method may be used to treat, e.g., a human, wherein said human, e.g., is afflicted with Alzheimer's disease. In another embodiment, said human being treated has a genetic predisposition or environment exposure that increases the likelihood that said person will develop Alzheimer's disease. For example, said human has suffered a head injury and is treated with a compound or composition as described herein. In one embodiment, said human exhibits minimal cognitive impairment suggestive of early stage Alzheimer's disease. In another embodiment, said human has suffered a head injury and is treated with a compound or composition as described herein.

The invention also provides compounds and compositions useful, for example, in treating Alzheimer's disease wherein the compound, or one or more active agents in the composition, is capable of crossing the blood brain barrier, where such compounds/agents include pirinixic acid in an esterified form, and pirinixic acid conjugated to DHA.

The invention also provides a method for delivering to the brain a compound capable of modulating Aβ production and/or release. This delivery system achieves specific delivery of such compounds through conjugating the compounds with a polar lipid or other carrier, achieving effective intracerebral concentration of such compounds efficiently and with specificity.

The invention also provides a method of treatment comprising modulating the production and/or release of β-amyloid in a human in need of said treatment, said method comprising administering to said human a compound that can modulate the production and/or release of β-amyloid in a human, or a composition comprising such a compound.

The invention also provides a method of treatment comprising modulating the production and/or release of β-amyloid in a non-human mammal in need of said treatment, said method comprising administering to said non-human mammal a compound that can modulate the production and/or release of β-amyloid in a human, or a composition comprising such a compound.

The invention further provides compositions of matter comprising a biologically active compound capable of modulating Aβ production and/or release covalently linked to a polar lipid carrier molecule. Preferred embodiments also comprise a spacer molecule having two linker functional groups, wherein the spacer has a first end and a second end and wherein the lipid is attached to the first end of the spacer through a first linker functional group and the biologically active compound is attached to the second end of the spacer through a second linker functional group. In preferred embodiments, the biologically active compound is a PPARα and/or PPARδ agonist. Preferred polar lipids include but are not limited to acyl- and acylated carnitine, sphingosine, ceramide, phosphatidyl choline, phosphatidyl glycerol, phosphatidyl ethanolamine, phosphatidyl inositol, phosphatidyl serine, cardiolipin and phosphatidic acid.

In one embodiment, the compound/agent in the methods of the invention is a compound of the formula

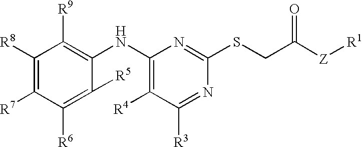

wherein, independently at each occurrence, R¹ is an organic moiety having at least 4 carbons; Z is selected from —O—, —NH—NH—, and —N(R²)—; R² is selected from hydrogen and C₁-C₃₀ organic moieties with the proviso that R¹ and R² can join together with the nitrogen to which they are both attached and form a heterocyclic moiety; R³ and R⁴ are each independently selected from the group consisting of hydrogen, halogen, lower alkyl and lower alkoxy radicals; R⁵, R⁶, R⁷, R⁸ and R⁹ are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R¹⁰—N═N—O—R¹¹, —OR¹², —C(O)OR¹², —N(R¹²)₂, —C(O)N(R¹²)₂, —N(R¹²)C(O)OR¹¹, heterocyclyl and heterocyclylalkyl; R¹⁰ is a bond or a straight or branched alkylene or alkenylene chain; R¹¹ is hydrogen, alkyl or aralkyl; and R¹² is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl. Preferably, Z is not NR² when R³ is Cl, R⁴ is H, R⁵ is H, R⁶ is H, R⁷ is H, R⁸ is methyl and R⁹ is methyl.

In another embodiment, the compound/agent in the methods of the invention is a compound of the formula

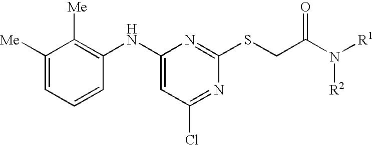

wherein, R¹ is a hydrophobic moiety selected from non-aromatic organic moieties having at least 10 carbon atoms and aromatic moieties having at least 6 carbons, and R² is hydrogen; or each of R¹ and R² are selected from hydrophobic organic moieties having at least one carbon atom, with the proviso that R¹ and R² in total have at least six carbon atoms, and with the further proviso that R¹ and R² can join together with the nitrogen to which they are both bonded and form a heterocyclic moiety.

In another embodiment, the compound/agent in the methods of the invention is a compound that (1) is a PPARα agonist and/or a PPARδ agonist, and (2) regulates the production and/or release of β-amyloid in cells.

In another embodiment, the compound/agent in the methods of the invention is a compound of the formula

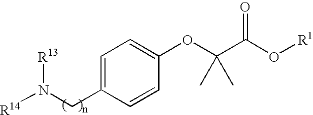

wherein, R¹ is an organic moiety having at least 4 carbons; R¹³ and R¹⁴ are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R¹⁰—N═N—O—R¹¹, —OR¹², —C(O)OR¹², —N(R¹²)₂, —C(O)N(R¹²)₂, —N(R¹²)C(O)OR¹¹, heterocyclyl and heterocyclylalkyl; R¹⁰ is a bond or a straight or branched alkylene or alkenylene chain; R¹¹ is hydrogen, alkyl or aralkyl; and R¹² is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl; and n is 1, 2 or 3.

In another embodiment, the compound/agent in the methods of the invention is a compound of the formula

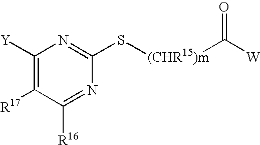

wherein, independently at each occurrence, R¹⁵ and R¹⁷ are each independently selected from the group consisting of hydrogen and lower alkyl radicals; R¹⁶ is selected from the group consisting of hydrogen, halogen and lower alkoxy radicals; W is selected from the group consisting of hydroxy, lower alkoxy, —OM and —(NH)ₚNH₂ radicals, wherein p is 0 or 1, and M is an alkali metal cation, an alkaline earth metal cation or the ammonium ion; m is 0, 1, 2 or 3; Y is selected from the group consisting of an aryl radical of 6 to 10 carbon atoms;

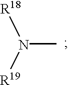

and

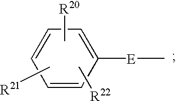

wherein R¹⁸ is hydrogen or lower alkyl radical; R¹⁹ is hydrogen, H₂N—,

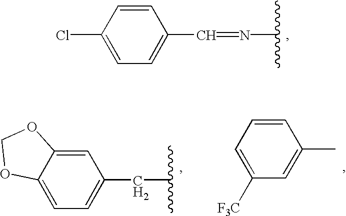

phenyl, (lower)alkoxyphenyl, or di(lower)alkoxy-phenyl, providing that when R¹⁸ is hydrogen and R¹⁹ is hydrogen, phenyl, (lower)alkoxyphenyl or di(lower)alkoxyphenyl, R¹⁶ is halo or lower alkoxy, R²⁰ is selected from the group consisting of a lower alkyl radical, a halo radical, an aryl radical of 6 to 10 carbon atoms and a haloaryl radical of 6 to 10 carbon atoms; R²¹ is selected from the group consisting of hydrogen, lower alkyl, lower alkoxy and halo radicals; R²² is selected from the group consisting of hydrogen and lower alkyl radicals; and E is selected from the group consisting of

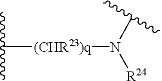

and

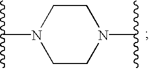

wherein R²³ is hydrogen or lower alkyl, R²⁴ is hydrogen or lower alkyl, and q is an integer from 0 to 3.

In additional embodiments, the invention provides the compounds as described herein, and compositions containing the compounds described herein.

These and related aspects of the present invention are described in further detail below.

### BRIEF DESCRIPTION OF THE SEVERAL VIEWS OF THE DRAWINGS

FIG. 1 is a bar graph showing the effect of PPARα and/or PPARδ agonist pirinixic acid on production and/or release of Aβ-40 and Aβ-42 from SM-4 cells. Cells were treated with 10-500 μM pirinixic acid. After 16 hr, the culture media was harvested and assayed for extracellular levels of Aβ-40 and Aβ-42 by ELISA. Extracellular Aβ was standardized to propridium iodide fluorescence as a measure of total cell number. Data are expressed as mean±SD with n=3-13 and statistical significance determined by ANOVA with Tukey's post hoc test at ***p<0.001. Double hatched bars indicate Aβ-40 levels and hatched bars indicate Aβ-42 levels.

FIG. 2 is a bar graph showing the effect of Clofibrate on levels of extracellular levels of Aβ-40 and Aβ-42 from SM-4 cells. Cells were treated with 10-500 μM Clofibrate. After 16 hrs, the culture media was harvested and assayed for extracellular Aβ-40 and Aβ-42 by ELISA. Secreted Aβ was standardized to propridium iodide fluorescence as a measure of total cell number. Data are expressed as mean±SD with n=5 and statistical significance determined by ANOVA with Tukey's post hoc test at ***p<0.001. Double hatched bars represent Aβ-40 levels as a percent of vehicle, hatched bars represent Aβ-42 levels as a percent of vehicle.

FIG. 3 is a bar graph showing the effect of ETYA on levels of extracellular levels of Aβ-40 and Aβ-42 from SM-4 cells. Cells were treated with 5-100 μM ETYA. After 16 hrs, the culture media was harvested and assayed for extracellular Aβ-40 and Aβ-42 by ELISA. Secreted Aβ was standardized to propridium iodide fluorescence as a measure of total cell number. Data are expressed as mean±SD with n=5 and statistical significance determined by ANOVA with Tukey's post hoc test at *p<0.05 and **p<0.01. Double hatched bars represent Aβ-40 levels as a percent of vehicle, and hatched bars represent Aβ-42 levels as a percent of vehicle.

FIG. 4 is a representative micrograph (upper panel) and a bar graph (lower panel) showing the effect of PPARα and/or PPARδ agonist pirinixic acid on cellular APP levels from SM-4 cells. Cells were treated with 50-500 μM pirinixic acid for 16 hours and cellular APP was quantitated by Western blot analysis. Data are expressed as mean±SD with n=4 and statistical significance determined by ANOVA with Tukey's post hoc test at *p<0.05 and **p<0.01.

FIG. 5 is a representative micrograph (upper panel) and a bar graph (lower panel) showing the effect of PPARα and/or PPARδ agonist pirinixic acid on APP_{sα} release from SM-4 cells. Cells were treated with 50-500 μM pirinixic acid for 16 hours and APP_{sα} release was quantitated by Western blot analysis. Data are expressed as mean±SD with n=4 and statistical significance determined by ANOVA with Tukey's post hoc test at **p<0.01.

FIG. 6 is a representative micrograph (upper panel) and a bar graph (lower panel) showing the effect of PPARα and/or PPARδ agonist pirinixic acid on C99 levels from SM-4 cells. Cells were treated with 50-500 μM pirinixic acid for 16 hours and C99 was quantitated by Western blot analysis. Data are expressed as mean±SD with n=4 and statistical significance determined by ANOVA with Tukey's post hoc test at **p<0.01.

FIG. 7 is a bar graph showing the effect of PPARα and/or PPARδ agonist pirinixic acid on secreted Aβ-40 and Aβ-42 from human neuroblastoma cells. Cells were treated with 100-200 μM of pirinixic acid after transient transfection with Swedish mutant APP. After a 16-hour treatment, the culture media was harvested and assayed for Aβ-40 and Aβ-42 by ELISA as described in the Methods and Materials. Secreted Aβ was standardized to propridium iodide fluorescence as a measure of total cell number. Data are expressed as mean±SD with n=11 and statistical significance determined by ANOVA with Tukey's post hoc test at ***p<0.001.

FIG. 8 is a bar graph showing the effect of PPARα and/or PPARδ agonist pirinixic acid on Aβtotal and Aβ-42 from murine primary cortical neurons infected with APP 695. Cells were treated with 5-250 μM pirinixic acid for 16 hours and Aβtotal and Aβ-42 levels were quantitated by immunoprecipitation and ELISA, respectively. Data are expressed as mean±SD with n=6 and statistical significance determined by ANOVA with Tukey's post hoc test at **p<0.01, ***p<0.001.

### DETAILED DESCRIPTION OF THE INVENTION

The invention is based on the inventors' discovery that exposure of mammalian cells to certain PPARα and/or PPARδ agonists modulates, specifically decreases the production and/or release of Aβ, particularly Aβ-42, from the cells. Because not all PPARα and/or PPARδ agonists achieve this effect, the invention also provides methods and materials for screening these agonists and related compounds and derivatives to determine their suitability for modulating Aβ production and/or release in vivo. Certain derivatives of the agonists have enhanced ability to penetrate the blood-brain barrier.

The invention is also based on the discovery that certain chemical compounds previously shown to decrease cholesterol levels have an effect on production and/or release of Aβ-42. The compounds include those of the general formula (I):

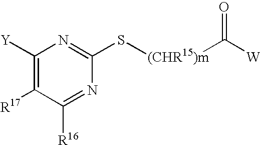

where R¹⁵, R¹⁶, R¹⁷, Y, W and m are defined elsewhere herein, where such compounds are exemplified by pirinixic acid with the structure:

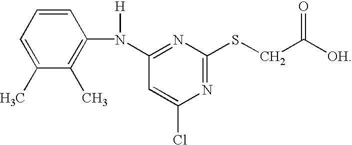

This invention discloses, for the first time, the use of these compounds and derivatives thereof to decrease β-amyloid production and/or release from cells, specifically the 42-amino acid form, Aβ-42, which has been implicated in the development and progression of Alzheimer's disease (AD). A connection exists between serum cholesterol levels and the incidence and the pathophysiology of AD, so the use of compounds that are known to be involved with the lowering of cholesterol may be effective in treating, preventing, and reducing the risk of AD. However, the present inventors have found that the cholesterol-lowering effect alone does not indicate that a compound will have an effect on Aβ production and/or release. Accordingly, the invention provides methods for selecting agents that have this desired effect on β-amyloid. One such group of compounds are agonists for members of the family of the peroxisome proliferator-activated receptors (PPAR), particularly PPARα and PPARδ.

1. PPARα and PPARδ Agonists

The peroxisome proliferator-activated receptors (PPARs) [α,δ, β, and γ] are a subfamily of the nuclear receptor gene family (reviewed in Desvergne & Wahli, Endocrine Rev 20:649-688 (1999)). All PPARs are, to various extents, activated by fatty acids and derivatives; PPARα binds the hypolipidemic fibrates whereas antidiabetic glitazones are ligands for PPARδ. PPARα activation mediates pleiotropic effects such as stimulation of lipid oxidation, alteration in lipoprotein metabolism and inhibition of vascular inflammation, to name but a few. PPARα activators increase hepatic uptake and the esterification of free fatty acids by stimulating the fatty acid transport protein and acyl-CoA synthetase expression. In skeletal muscle and heart, PPARα increases mitochondrial free fatty acid uptake and the resulting free fatty acid oxidation through stimulating the muscle-type carnitine palmitoyltransferase-I. The effect of fibrates on the metabolism of triglyceride-rich lipoproteins is due to a PPARα dependent stimulation of lipoprotein lipase and an inhibition of apolipoprotein C-III expression, whereas the increase in plasma HDL cholesterol depends on an overexpression of apolipoprotein A-I and apolipoprotein A-II.

In contrast to PPARα, the function of PPARδ is not well understood. Although PPARδ is ubiquitously expressed the brain, adipose tissue and skin have higher levels of relative mRNA expression (Peters, J. M. et al., Mol. Cell. Biol. 20:5119-5128, 2000). Based on its expression profile, Xing G., et al. (Biochem. Biophys. Res. Commun. 217:1015-1025, 1995) suggest that PPARδ may be involved in brain functions. Furthermore, PPARδ may be implicated in reverse cholesterol transport (Oliver, W. R. et al., Proc. Nat'l. Acad. Sci. 98:5306-5311, 2001). Examples of PPARδ agonists include but are not limited to valproic Acid (Lampen et al., Tox. Appl. Pharmacol. 160:238-249, 1999), GW501516 (Oliver, W. R. et al., Proc. Nat'l. Acad. Sci. 98:5306-5311, 2001), L-165041, L-165461, L-783483, and L-796449 (Berger et al., J. Biol. Chem. 274:6718-6725, 1999).

For example, the invention provides a method of treatment comprising modulating the production and/or release of β-amyloid in a human in need of said treatment, said method comprising administering to said human a compound of the formula

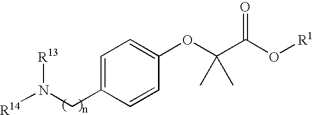

wherein, R¹ is selected from the group consisting of C₁-C₃ alkyl, hydrogen, metal cation and ammonium cation; R¹³ and R¹⁴ are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R¹⁰—N═N—O—R¹¹, —OR¹², —C(O)OR¹², —N(R¹²)₂, —C(O)N(R¹²)₂, —N(R¹²)C(O)OR¹¹, heterocyclyl and heterocyclylalkyl; R¹⁰ is a bond or a straight or branched alkylene or alkenylene chain; R¹¹ is hydrogen, alkyl or aralkyl; R¹² is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl; and n is 1, 2 or 3.

Specific compounds having PPARα agonist and/or PPARδ agonist activity are compounds having the formula

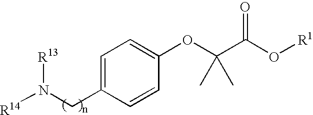

wherein, in one embodiment, R¹ is hydrogen, while in another embodiment R¹ is a metal cation or an ammonium cation, while in another embodiment R¹ is an organic moiety having at least 2, or at least 3, or at least 4, or at least 5, or at least 6 carbons; while in another embodiment R¹ enhances the penetration of the compound through the blood brain barrier relative to the corresponding compound wherein R¹ is hydrogen, R¹³ and R¹⁴ are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R¹⁰—N═N—O—R¹¹, —OR¹², —C(O)OR¹², —N(R¹²)₂, —C(O)N(R¹²)₂, —N(R¹²)C(O)OR¹¹, heterocyclyl and heterocyclylalkyl; R¹⁰ is a bond or a straight or branched alkylene or alkenylene chain; R¹¹ is hydrogen, alkyl or aralkyl; R¹² is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl; and n is 1, 2 or 3. In various embodiments, R¹ is an organic group having less than 30 carbons and a formula weight of less than 1,000, or less than 900, or less than 800, or less than 700, or less than 600, or less than 500. In addition, or alternatively, R¹ can be described as being hydrophobic. In addition, or alternatively, R¹ is selected from the group consisting of alkyl, alkenyl, aryl, aralkyl, aralkenyl, cycloalkyl, cycloalkylalkyl, cycloalkylalkenyl, halogen, haloalkyl, haloalkenyl, cyano, nitro, —R¹⁰—N═N—O—R¹¹, —OR¹², —C(O)OR¹², —N(R¹²)₂, —C(O)N(R¹²)₂, —N(R¹²)C(O)OR¹¹, heterocyclyl and heterocyclylalkyl. In addition, or alternatively, R¹ is a straight-chained hydrocarbon moiety containing between 16 and 26 carbon atoms, wherein the moiety is selected from the group consisting of C16:0; C16:1; C16:2; C20:1; C20:2; C20:3; C20:4; C22:4; C22:5; C22:6 and C24:4. In addition, or alternatively, R¹ is a fragment of insulin wherein said insulin fragment binds to an insulin receptor, for example, said fragment of insulin may consist of: (a) a peptide chain having 14 to 21 amino acid residues from the N-terminus of insulin chain A; and (b) another peptide chain having 16 to 22 amino acid residues from the N-terminus of insulin chain B. In addition, or alternatively, R¹ is a protein that binds to a transferrin receptor. In addition, or alternatively, R¹ is an antibody or a fragment thereof capable of binding to a ligand in the brain, for example, said antibody may be a monoclonal antibody. In addition, or alternatively, R¹ is a growth factor, for example, said growth factor may be EGF.

Other exemplary PPARα agonists consist of the following structure:

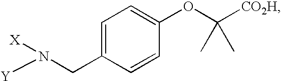

wherein X is selected from the group (a-t) as shown below, and Y is selected from the group (1-8) as shown below.

Exemplary PPARδ agonists consist of the following structure:

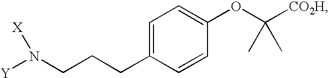

wherein X is selected from the group (a-t) as shown below, and Y is selected from the group (1-8) as shown below.

| X | | | | Y | |
|---|---|---|---|---|---|
| a | 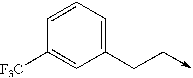 | k | 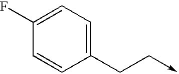 | 1 | 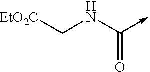 |
| b | 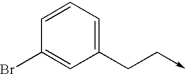 | l | 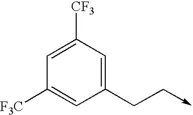 | 2 | 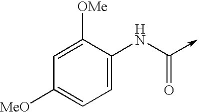 |
| c | 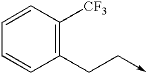 | m | 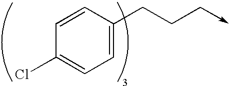 | 3 | 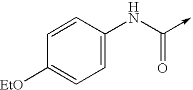 |
| d | 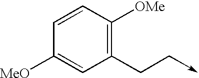 | n | 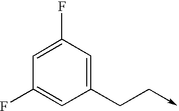 | 4 | 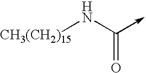 |
| e | 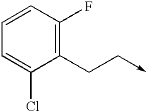 | o | 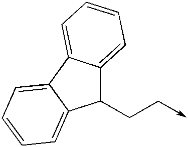 | 5 | 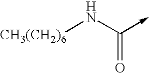 |
| f | 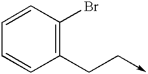 | p | 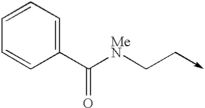 | 6 | 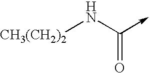 |
| g | 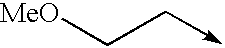 | q | 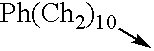 | 7 | 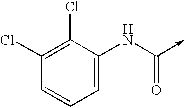 |
| h | 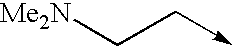 | r | 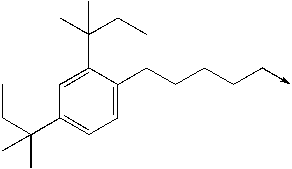 | 8 | 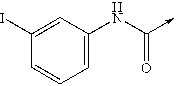 |
| i | 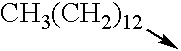 | s | 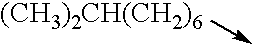 | | |
| j | 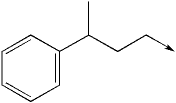 | t | 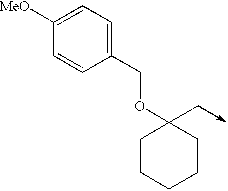 | | |

A preferred member of this group of agonists has the formula

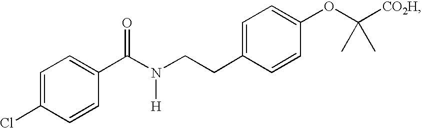

and is also referred to as bezafibrate (Brown, P. J. et al., Chem. And Biol. 4:909-918, 1997), where this compound or esters thereof, i.e., the carboxylic acid of bezafibrate or a reactive equivalent thereof is reacted with an alcohol or a reactive equivalent thereof to form the corresponding ester having an R¹ group, may be used in the methods of the present invention.

Another preferred compound, a PPARδ agonist also disclosed by Brown, P. J. et al., is referred to as 9w2433 and has the following structure:

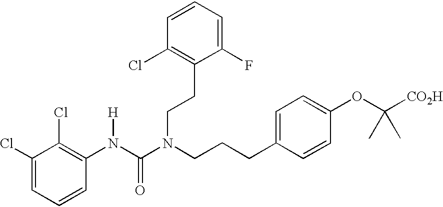

where 9w2433 and esters thereof, i.e., the carboxylic acid of 9w2433 or a reactive equivalent thereof is reacted with an alcohol or a reactive equivalent thereof to form the corresponding ester having an R¹ group, are preferred compounds, and are preferred agents in the methods and compositions disclosed herein.

PPARs are also expressed in atherosclerotic lesions (Bishop-Bailey, Br. J. Pharmacol. 129:823-834, 2000). PPARα is present in endothelial and smooth muscle cells, monocytes and monocyte-derived macrophages. It inhibits inducible nitric oxide synthase in macrophages and prevents the IL-1-induced expression of IL-6 and cyclooxygenase-2, as well as thrombin-induced endothelin-1 expression, as a result of a negative transcriptional regulation of the nuclear factor-kappa B and activator protein-1 signaling pathways. PPAR activation also induces apoptosis in human monocyte-derived macrophages, most likely through inhibition of nuclear factor-kappa B activity. Therefore, the pleiotropic effects of PPARα activators on the plasma lipid profile and vascular wall inflammation likely participate in the inhibition of atherosclerosis development. In addition to lowering cholesterol, according to the present invention, they may also be effective in treating, preventing, and reducing the risk of AD.

2. Pirinixic Acid and Analogs and Derivatives Thereof

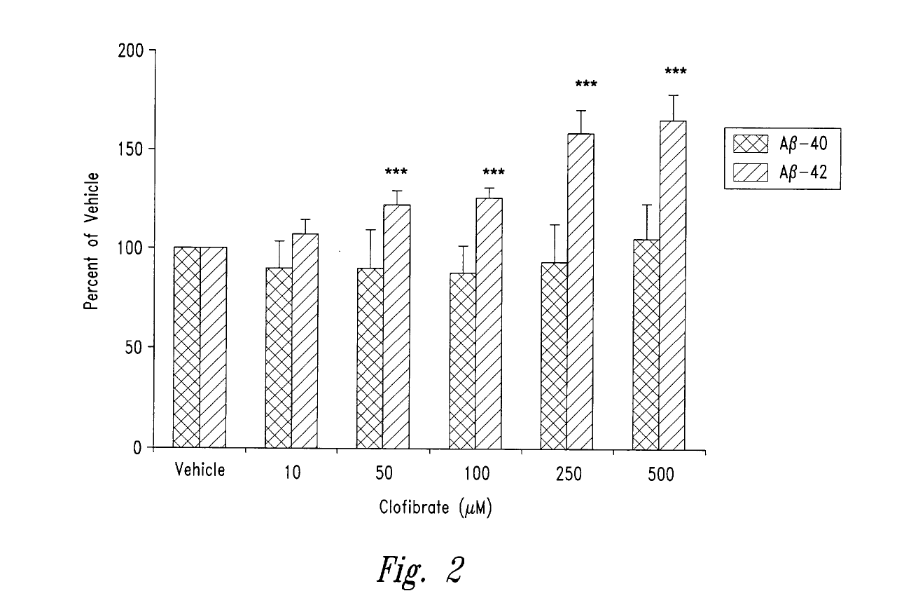

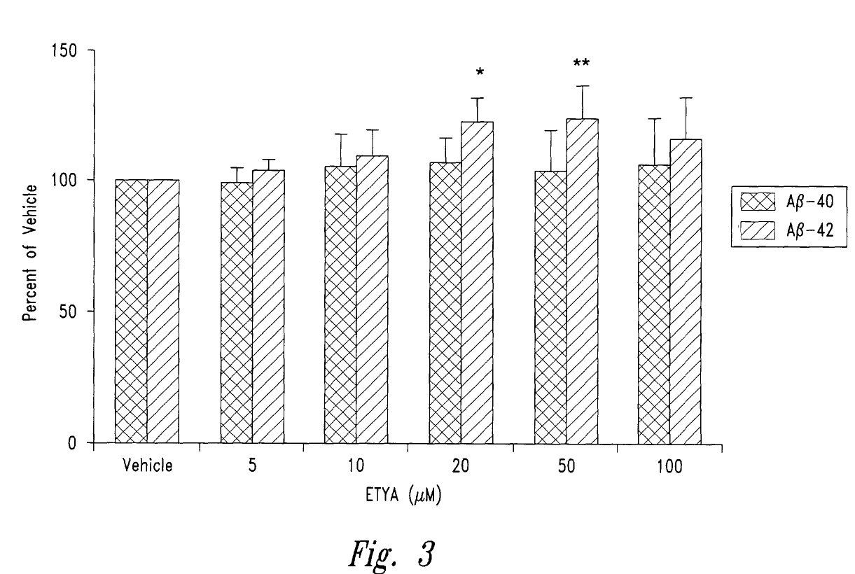

The invention therefore provides PPARα and/or PPARδ agonists and derivatives thereof for use in lowering β-amyloid levels, and thereby alleviating, treating, and/or preventing disease associated with buildup of β-amyloid, such as Alzheimer's disease. According to the invention, an exemplary PPARα agonist, pirinixic acid, is useful in reducing Aβ-42 production and/or release from cells. By inhibiting Aβ-42 production and/or release, buildup of Aβ-42 and formation of plaques may be reduced or prevented. These results are consistent with current models for the role of Aβ in Alzheimer's disease. However, not all PPARα agonists can be used for lowering β-amyloid production and/or release. For example, the PPARα agonists ETYA and Clofibrate were found to increase the production and/or release of the Aβ-42 from cells, as shown in FIGS. 2 and 3 and as discussed in detail in the examples. These results demonstrate that the definition of a compound as a PPARα agonist is not the only factor that determines an efficacious response (i.e., a decrease in Aβ production and/or release). Rather, the response appears to be specific to the chemical structure. A novel aspect of the invention is the provision of methods and materials for screening PPARα and/or PPARδ agonists and related compounds and derivatives to determine their suitability for modulating Aβ production and/or release from cells in vivo.

The invention also relates to the use of compounds, and pharmaceutical compositions containing said compounds, having the (2-pyrimidinylthio) alkanoic acid, ester, amide and hydrazide structures of the structural formula:

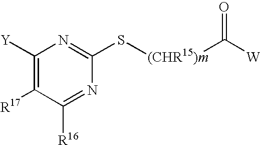

wherein, independently at each occurrence, R¹⁵ and R¹⁷ are each independently selected from the group consisting of hydrogen and lower alkyl radicals; R¹⁶ is selected from the group consisting of hydrogen, halogen and lower alkoxy radicals; W in one embodiment is hydrogen while in another embodiment W is selected from the group consisting of hydroxyl, lower alkoxy, —OM and —(NH)ₚNH₂ radicals, wherein p is 0 or 1, and M is an alkali metal cation, an alkaline earth metal cation or the ammonium ion; m is 0, 1, 2 or 3; Y is selected from the group consisting of an aryl radical of 6 to 10 carbon atoms;

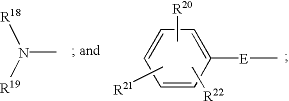

wherein R¹⁸ is hydrogen or lower alkyl radical; R¹⁹ is hydrogen, H₂N—,

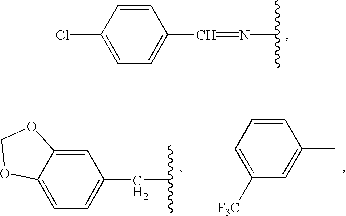

phenyl, (lower)alkoxyphenyl, or di(lower)alkoxy-phenyl, providing that when R¹⁸ is hydrogen and R¹⁹ is hydrogen, phenyl, (lower)alkoxyphenyl or di(lower)alkoxyphenyl, R¹⁶ is halo or lower alkoxy; R²⁰ is selected from the group consisting of a lower alkyl radical, a halo radical, an aryl radical of 6 to 10 carbon atoms and a haloaryl radical of 6 to 10 carbon atoms; R²¹ is selected from the group consisting of hydrogen, lower alkyl, lower alkoxy and halo radicals; R²² is selected from the group consisting of hydrogen and lower alkyl radicals, and E is selected from the group consisting of

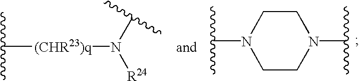

wherein R²³ is hydrogen or lower alkyl; R²⁴ is hydrogen or lower alkyl; and q is an integer from 0 to 3.

Said compounds are exemplified by pirinixic acid with the structure:

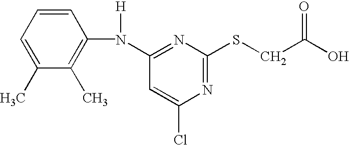

as well as esters, etc. thereof.

In one aspect, the pirinixic acid derivative compounds have the formula

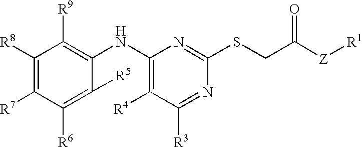

wherein, independently at each occurrence, R¹ is hydrogen in one embodiment, while R¹ is an organic moiety having at least 1, at least 2, at least 3, at least 4 carbons, and at least 5 in various additional embodiments; Z is selected from —O—, —NH—NH—, and —N(R²)—; R² is selected from hydrogen and C₁-C₃₀ organic moieties with the proviso that R¹ and R² can join together with the nitrogen to which they are both attached and form a heterocyclic moiety; R³ and R⁴ are each independently selected from the group consisting of hydrogen, halogen, lower alkyl and lower alkoxy radicals; R⁵, R⁶, R⁷, R⁸ and R⁹ are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R¹⁰—N═N—O—R¹¹, —OR¹², —C(O)OR¹², —N(R¹²)₂, —C(O)N(R¹²)₂, —N(R¹²)C(O)OR¹¹, heterocyclyl and heterocyclylalkyl; R¹⁰ is a bond or a straight or branched alkylene or alkenylene chain; R¹¹ is hydrogen, alkyl or aralkyl; and R¹² is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl. Optionally, these compounds are described with the proviso that Z is not NR² when R³ is Cl, R⁴ is H, R⁵ is H, R⁶ is H, R⁷is H, R⁸ is methyl and R⁹ is methyl.

In another aspect, the pirinixic acid derivative compounds are amides of pirinixic acid having the formula

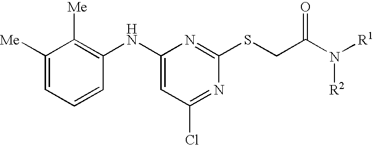

wherein R¹ and R² are hydrogen or organic moieties. In various embodiments of the invention, one, two or more of the following criteria may be further applied to describe compounds of this formula, where any two or more criteria may be combined so long as those criteria are not inconsistent with one another: R¹ is aromatic, R¹ is non-aromatic, R¹ is aliphatic, R¹ has no more than 30 carbon atoms, R¹ has no more than 25 carbon atoms, R¹ has no more than 20 carbon atoms, R¹ has at least 2 carbon atoms, R¹ has at least 3 carbon atoms, R¹ has at least 4 carbon atoms, R¹ has at least 5 carbon atoms, R¹ has at least 6 carbon atoms, R¹ has at least 7 carbon atoms, R¹ has at least 8 carbon atoms, R¹ has at least 9 carbon atoms, R¹ has at least 10 carbon atoms, R¹ has a formula weight of less than 1,000; R¹ has a formula weight of less than 900, R¹ has a formula weight of less than 800, R¹ has a formula weight of less than 700, R¹ has a formula weight of less than 600, R¹ has a formula weight of less than 500, R¹ has a formula weight of less than 400, R¹ is alkyl, R¹ is alkenyl, R¹ is aryl, R¹ is aralkyl, R¹ is aralkenyl, R¹ is cycloalkyl, R¹ is cycloalkylalkyl, R¹ is cycloalkylalkenyl, R¹ is halogen, R¹ is haloalkyl, R¹ is haloalkenyl, R¹ is cyano, R¹ is nitro, R¹ is R¹⁰—N═N—O—R¹¹, R¹ is —OR¹², R¹ is —C(O)OR¹², R¹ is —N(R¹²)₂, R¹ is —C(O)N(R¹²)₂, R¹ is —N(R¹²)C(O)OR¹¹, where R¹⁰, R¹¹ and R¹² are defined elsewhere herein, R¹ is heterocyclyl, R¹ is heterocyclylalkyl, R¹ is a hydrocarbon, R¹ is a straight-chained hydrocarbon moiety containing between 16 and 26 carbon atoms, wherein the moiety is selected from the group consisting of C16:0; C16:1; C16:2; C20:1; C20:2; C20:3; C20:4; C22:4; C22:5; C22:6 and C24:4; R¹ is a fragment of insulin wherein said insulin fragment binds to an insulin receptor, e.g., R¹ is a fragment of insulin that consists of (a) a peptide chain having 14 to 21 amino acid residues from the N-terminus of insulin chain A; and (b) another peptide chain having 16 to 22 amino acid residues from the N-terminus of insulin chain B; R¹ is a protein that binds to a transferrin receptor; R¹ is an antibody or a fragment thereof capable of binding to a ligand in the brain, e.g., R¹ is a monoclonal antibody; R¹ is a growth factor, e.g., EGF; R¹ imparts to the compound the property of enhanced penetration of the blood brain barrier relative to the corresponding compound wherein R¹ is hydrogen, R² is hydrogen, R² is selected from groups that R¹ may be as defined above, R¹ and R² can join together with the nitrogen to which they are both bonded and form a heterocyclic moiety, R¹ and R² in total have at least 2, or at least 3, or at least 4, or at least 5, or at least 6 carbons. For example, in one embodiment, R¹ is a hydrophobic moiety selected from non-aromatic organic moieties having at least 10 carbon atoms and aromatic moieties having at least 6 carbons, and R² is hydrogen. As another example, in another embodiment, each of R¹ and R² are selected from hydrophobic organic moieties having at least one carbon atom, with the proviso that R¹ and R² in total have at least six carbon atoms, and with the further proviso that R¹ and R² can join together with the nitrogen to which they are both bonded and form a heterocyclic moiety.

An exemplary composition useful in the methods of the present invention comprises pirinixic acid and derivatives thereof as described herein, with an pharmaceutically acceptable carrier, diluent or excipient.

By the expression “lower,” used to modify the terms alkyl and alkoxy, applicants mean to limit the aliphatic chain length of those monovalent, branched and unbranched groups of paraffinic derivation to from 1 to 6 carbon atoms. By the term “halo” or “halogens” applicants mean to embrace chlorine, fluorine, iodine and bromine.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In one embodiment, a straight chain or branched chain alkyl has 30 or fewer carbon atoms in its backbone (e.g., C₁-C₃₀ for straight chain, C₃-C₃₀ for branched chain), and more preferably 20 or fewer. Likewise, cycloalkyls have from 4-10 carbon atoms in their ring structure, and more preferably have 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxy alkoxycarbonyloxy, arloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, aralkyl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (benzyl)).

Unless the number of carbons is otherwise specified, “lower alkyl” as used herein means an alkyl group, as defined above, but having from one to ten carbons, more preferably from one to six carbon atoms in its backbone structure. Preferred alkyl groups are lower alkyls having one to three carbon atoms.

The term “aryl” refers to a phenyl or naphthyl radical. Unless stated otherwise specifically in the specification, the term “aryl” or the prefix “ar-” (such as in “aralkyl”) is meant to include phenyl and naphthyl radicals optionally substituted by one or more substituents as described above in connection with the term “alkyl”. In one embodiment of the invention, the aryl group is phenyl. In another or additional embodiment, the aryl group has a single substituent. In another or additional embodiment, the aryl group has two substituents.

The term “cycloalkyl” refers to a stable monovalent monocyclic or bicyclic hydrocarbon radical consisting solely of carbon and hydrogen atoms, having from three to ten carbon atoms, and which is saturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents independently selected from the group of substituents identified above in connection with the “alkyl” groups. In one embodiment, the alkyl group is mono-substituted. In another embodiment, the alkyl group is unsubstituted.

The term “heterocyclyl” refers to a stable 3- to 15-membered ring radical which consists of carbon atoms and from one to five heteroatoms selected from the group consisting of nitrogen, oxygen and sulfur. For purposes of this invention, the heterocyclyl radical may be a monocyclic, bicyclic or tricyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be aromatic or partially or fully saturated. The heterocyclyl radical may not be attached to the rest of the molecule at any heteroatom atom. Examples of such heterocyclyl radicals include, but are not limited to, azepinyl, acridinyl, benzimidazolyl, benzthiazolyl, benzothiadiazolyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,5]imidazo[1,2-a]pyridinyl; carbazolyl, cinnolinyl, dioxolanyl, decahydroisoquinolyl, furanyl, furanonyl, isothiazolyl, imidazolyl, imidazolinyl, imidazolidinyl, isothiazolidinyl, indolyl, indazolyl, isoindolyl, indolinyl, isoindolinyl, indolizinyl, isoxazolyl, isoxazolidinyl, morpholinyl, naphthyridinyl, oxadiazolyl, octahydroindolyl, octahydroisoindolyl, 2-oxopiperazinyl, 2-oxopiperidinyl, 2-oxopyrrolidinyl, 2-oxoazepinyl, oxazolyl, oxazolidinyl, oxiranyl, piperidinyl, piperazinyl, 4-piperidonyl, phenazinyl, phenothiazinyl, phenoxazinyl, phthalazinyl, pteridinyl, purinyl, pyrrolyl, pyrrolidinyl, pyrazolyl, pyrazolidinyl, pyridinyl, pyrazinyl, pyrimidinyl, pyridazinyl, quinazolinyl, quinoxalinyl, quinolinyl, quinuclidinyl, isoquinolinyl, thiazolyl, thiazolidinyl, thiadiazolyl, triazolyl, tetrazolyl, tetrahydrofuryl, triazinyl, tetrahydropyranyl, thienyl, thiamorpholinyl, thiamorpholinyl sulfoxide, and thiamorpholinyl sulfone. Unless stated otherwise specifically in the specification, the term “heterocyclyl” is meant to include heterocyclyl radicals as defined above which are optionally substituted by one or more substituents as defined above in connection with the description of “alkyl” groups. In one embodiment of the invention, the heterocyclic group does not have a substituent. In another embodiment, the heterocyclic group has a single substituents.

In various embodiments, as to the pirinixic acid derivatives and analog compounds identified herein, one or more of the following criteria may be applied in order to further define the compounds of interest, where any two or more criteria may be combined together so long as no two of the criteria are inconsistent with one another: Z is —O—, Z is —NH—NH—, Z is —N(H)—, Z is —N(R²)—, R¹ is an organic group having less than 30 carbons, R¹ is an organic group having less than 25 carbons, R¹ is an organic group having less than 20 carbons, R¹ is an organic group having less than 15 carbons, R¹ is an organic group having at least 2 carbons, R¹ is an organic group having at least 3 carbons, R¹ is an organic group having at least 4 carbons, R¹ is an organic group having at least 5 carbons, R¹ is an organic group having at least 6 carbons, R¹ has a formula weight of less than 1,000; R¹ has a formula weight of less than 900, R¹ has a formula weight of less than 800, R¹ has a formula weight of less than 700, R¹ has a formula weight of less than 600, R¹ has a formula weight of less than 500, R¹ has a formula weight of less than 400, R¹ is alkyl, R¹ is alkenyl, R¹ is aryl, R¹ is aralkyl, R¹ is aralkenyl, R¹ is cycloalkyl, R¹ is cycloalkylalkyl, R¹ is cycloalkylalkenyl, R¹ is halogen, R¹ is haloalkyl, R¹ is haloalkenyl, R¹ is cyano, R¹ is nitro, R¹ is R¹⁰—N═N—O—R¹¹, R¹ is —OR¹², R¹ is —C(O)OR¹², R¹ is —N(R¹²)₂, R¹ is —C(O)N(R¹²)₂, R¹ is —N(R¹²)C(O)OR¹¹, R¹ is heterocyclyl, R¹ is heterocyclylalkyl, R¹ is a straight-chained hydrocarbon moiety containing between 16 and 26 carbon atoms, wherein the moiety is selected from the group consisting of C16:0; C16:1; C16:2; C20:1; C20:2; C20:3; C20:4; C22:4; C22:5; C22:6 and C24:4; R¹ is a fragment of insulin wherein said insulin fragment binds to an insulin receptor, e.g., R¹ is a fragment of insulin that consists of (a) a peptide chain having 14 to 21 amino acid residues from the N-terminus of insulin chain A; and (b) another peptide chain having 16 to 22 amino acid residues from the N-terminus of insulin chain B; R¹ is a protein that binds to a transferrin receptor; R¹ is an antibody or a fragment thereof capable of binding to a ligand in the brain, e.g., R¹ is a monoclonal antibody; R¹ is a growth factor, e.g., EGF; R⁵ is hydrogen; R⁵ is halogen; R⁵ is lower alkyl; R⁵ is lower alkoxy; R⁶ is hydrogen; R⁶ is halogen; R⁶ is lower alkyl; R⁶ is lower alkoxy; R⁷ is hydrogen; R⁷ is halogen; R⁷ is lower alkyl; R⁷ is lower alkoxy; R⁸ is hydrogen; R⁸ is halogen; R⁸ is lower alkyl; R⁸ is lower alkoxy; R⁹ is hydrogen; R⁹ is halogen; R⁹ is lower alkyl; R⁹ is lower alkoxy; R¹ imparts to the compound the property of enhanced penetration of the blood brain barrier relative to the corresponding compound wherein R¹ is hydrogen.

The Aβ-modulating compounds used according to this invention may be readily prepared from (4,6-dichloro-2-pyrimidinylthio) alkanoic acid intermediates which themselves are obtained, for example, by converting 2-thiobarbituric acid to the (4,6-dihydroxy-2-pyrimidinythio)alkanoic acid ester by reaction with an alpha-halo (lower)alkanoic acid ester and subsequently displacing the 4- and 6-positioned hydroxyl groups with chlorine by reaction with an agent such as POCl₃, PCl₅, and the like. For instance:

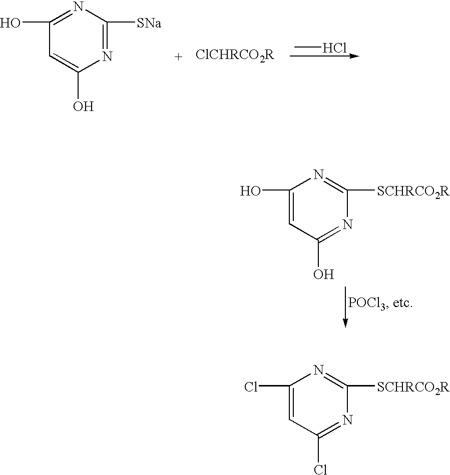

Various modifications of the 4,6-halo groups may be accomplished by substitution and displacement reactions. Thus, reactions of the (4,6-dichloro-2-pyrimidinylthio)alkanoic acid esters with primary amines yields the corresponding 4- or 6-amino derivative, reaction with hydrazine affords the 4- or 6-hydrazino derivative which readily converted to a hydrazone by reaction with an aldehyde or a carbonhydrazide by reaction with a carboxylic acid halide. An aryl group is positioned directly on the 4- or 6-position of the pyrimidine nucleus, if desired, by employing 6-phenyl-2-thiouracil as the initial reactant in lieu of a thio-barbituric acid. From the intermediate monochloro-4 or 6-substituted-2-pyrimidinylthio acetic acid ester, modification of the carboxylic acid functional group is readily achieved by transesterification, saponification and hydrolysis as well as by amidation of the free carboxyl group or the corresponding acid halide.

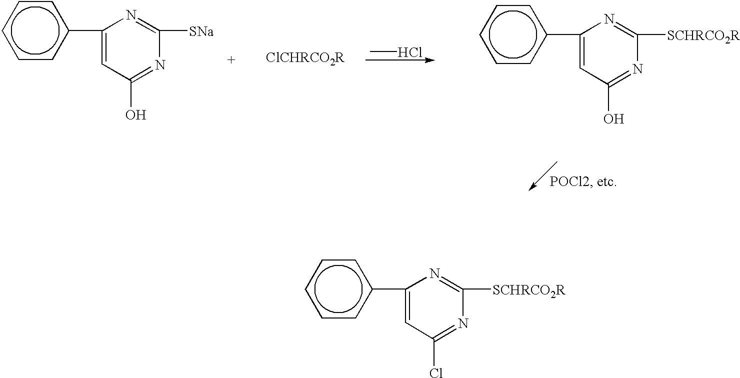

The compounds are administered to an individual suffering from Alzheimer's disease in unit doses containing from 0.05 to 25 milligrams of active ingredient, the remainder of the formulation constituting known adjuvants. The goal of the therapy is modulation of amyloid production and/or release. This modulation can be by one or more chemically induced physiological mechanisms.

The term “subject” is intended to include mammals having amyloid production and/or release, including one or more amyloid related symptoms, or which are susceptible to amyloid production and/or release. Examples of such subjects include humans, dogs, cats, pigs, cows, horses, rats and mice.

In human treatment, from 1 to 10 milligram and conventionally 5 milligram doses of the active compounds of this invention are considered to be most desirable from the standpoint of uniform presentation for controlled administration. The compounds of the invention may be administered alone or in combination with pharmacologically acceptable carriers, the proportion of which is determined by the chosen route of administration and standard pharmaceutical practice. For example, they may be administered orally in tablet or capsule form with conventional flavors, diluents, lubricants, disintegrators or binding agents as may be required. They may be administered orally in the form of a solution or they may be injected parenterally. For parenteral administration they may be used in the form of a sterile solution containing other solutes, for example, enough saline or glucose to make the solution isotonic.

A suitable tablet formulation is as follows:

| | |
|---|---|
| [4-chloro-6-(2,3-xylidino)-2-pyrimidinylthio] | |
| acetamide | .05 mg. |
| Microcrystalline cellulose, N.F. | .20 mg. |
| Magnesium stearate, U.S.P. | 25.00 mg. |
| Lactose, U.S.P. | 74.75 mg. |
| Total Weight | 100.00 mg. |

A suitable formulation for parenteral administration is as follows:

| | |
|---|---|
| Sodium[4-chloro-6-(2,3-xylidino)-2- | 5 mg |
| pyrimidinylthio]acetate | |
| Vehicle: sterile water, containing benzyl alcohol | 5 ml |
| (1 percent) and sodium acetate-acetic | |
| acid buffer 0.6% | |

Preferred compounds include those of the formula:

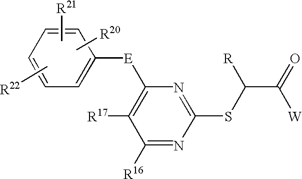

wherein, independently at each occurrence, R¹⁶ is selected from the group consisting of hydrogen and chloro radicals; R, R¹⁷ and R²² are independently selected from the group consisting of hydrogen and lower alkyl radicals; R²⁰ is selected from the group consisting of lower alkyl; lower alkoxy, aryl of 6 to 10 carbon atoms, haloaryl of 6 to 10 carbon atoms and halo radicals; R²¹ is selected from the group consisting of —H, lower alkyl, halo and lower alkoxy radicals; E is selected from the group consisting of

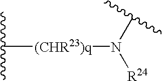

and

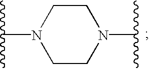

wherein R²³ and R²⁴ are independently —H or lower alkyl and q is an integer from 0 to 3, providing that when q is 0 and R²⁰ is lower alkoxy, R²¹ is lower alkyl, lower alkoxy or halo; and Z is selected from the group consisting of —OH, OM, lower alkoxy and —(NH)ₚ—NH₂, in which p is an integer from 0 to 1 and M is an alkali metal, alkaline earth metal or ammonium cation.

Preferred compounds are the [4-chloro-6-arylamino-2-pyrimidinylthio] acetic acid, alkali metal salt, amide, hydrazide and lower alkyl ester in which the aryl group contains from 7 to 12 carbon atoms, and the 6-para-chlorophenylamino and 6-para-chlorobenzylamino analogues thereof.

More preferred compounds of the invention may be represented by the following formula:

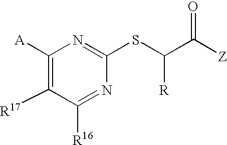

wherein, independently at each occurrence, A is a member selected from the group consisting of aryl of 6 to 10 carbon atoms and

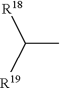

wherein R¹⁸ is —H or lower alkyl and R¹⁹ is hydrogen, H₂N—,

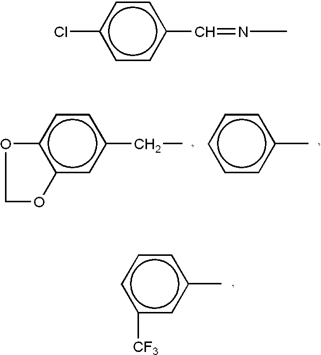

R is selected from the group consisting of —H and lower alkyl; R¹⁷ is selected from the group consisting of —H and lower alkyl; R¹⁶ is selected from the group consisting of —H, chloro and lower alkoxy radicals, with the proviso that when A is the amino or phenylamino group R¹ is chloro or lower alkoxy; and Z is selected from the group consisting of —NHNH₂, lower alkoxy, —OH and OM, wherein M is an alkali metal, alkaline earth metal or ammonium cation.

Specifically preferred compounds include:

(4,6-dichloro-2-pyrimidinylthio)acetic acid, ethyl ester.

(4-amino-6-chloro-2-pyrimidinylthio)acetic acid ethyl ester.

(4-anilino-6-chloro-2-pyrimidinylthio)acetic acid ethyl ester.

(4-chloro-6-(p-chloroanilino)-2-pyrimidinylthio)acetic acid ethyl ester.

[4-chloro-6-(p-fluoroanilino)-2-pyrimidinylthio]acetic acid ethyl ester.

[4-chloro-6-(α,α,α-trifluoro-m-toluidino)-2-pyrimidinylthio]acetic acid ethyl ester.

[4-chloro-6-(2,3-xylidino)-2-pyrimidinylthio]acetic acid ethyl ester.

[4-chloro-6-(2,4,6-trimethylanilino)-2-pyrimidinylthio]acetic acid ethyl ester.

[4-chloro-6-(p-methoxyanilino)-2-pyrimidinylthio]acetic acid ethyl ester.

[4-(4-biphenylylamino)-6-chloro-2-pyrimidinylthio]acetic acid ethyl ester.

(4-chloro-6-[4-(p-chlorophenyl)-1-piperazinyl]-2-pyrimidinylthio)acetic acid ethyl ester.

[4-chloro-6-(2,3-xylindino)-2-pyrimidinylthio]acetic acid.

[4-chloro-6-(2,3-xylindino)-2-pyrimidinylthio]acetamide.

[4-chloro-6-(2,3-xylidino)-2-pyrimidinylthio]acetic acid hydrazide.

[4-chloro-6-(p-chlorobenzylamino)-2-pyrimidinylthio]acetic acid, ethyl ester.

[4-chloro-6-(p-fluorobenzylamino)-2-pyrimidinylthio]acetic acid, ethyl ester.

[4-chloro-6-(3,4-dichlorobenzylamino)-2-pyrimidinylthio]acetic acid, ethyl ester.

[4-chloro-6-(2,4-dimethoxyanilino)-2-pyrimidinylthio]acetic acid.

[4-chloro-6-(2,4-dimethyqlbenzylamino)-2-pyrimidinylthio]acetic acid ethyl ester.

[4-chloro-6-(p-chlorophenethylamino)-2-pyrimidinylthio]acetic acid ethyl ester.

(4-chloro-6-[(p-chlorobenzyl)methylamino]-2-pyrimidinylthio)acetic acid ethyl ester.

[4-chloro-6-(p-chloro-α-methylbenzylamino)-2-pyrimidinylthio]acetic acid.

(4-chloro-6-[3,4-(methylenedioxy)benzylamino]-2-pyrimidinylthio))acetic acid ethyl ester.

[4-chloro-6-(p-chlorobenzylidenehydrazino)-2-pyrimidinylthio]acetic acid ethyl ester.

(4-chloro-6-[(p-fluorobenzylidene)hydrazino]-2-pyrimidinylthio)acetic acid ethyl ester.

(4-chloro-6-hydrazino-2-pyrimidinylthio)acetic acid, ethyl ester, hydrochloride.

[4-chloro-6-(p-chlorobenzylamino)-2-pyrimidinylthio]acetic acid.

(4-chloro-6-(p-chlorobenzylamino)-2-pyrimidinylthio)acetic acid hydrazide.

2-(4,6-dichloro-2-pyrimidinylthio)propionic acid ethyl ester.

2-[4-chloro-6-(p-chlorobenzylamino)-2-pyrimidinylthio]propionic acid.

(4-chloro-6-phenyl-2-pyrimidinylthio)acetic acid ethyl ester.

(4-methoxy-6-phenyl-2-pyrimidinylthio)acetic acid.

[4-(p-chlorobenzylamino)-2-pyrimidinylthio]acetic acid ethyl ester.

[4-(p-chlorobenzyl)methylamino-2-pyrimidinylthio]acetic acid ethyl ester,

(4,6-dichloro-5-methyl-2-pyrimidinylthio)acetic acid, ethyl ester.

[4-chloro-6-(p-chlorobenzylamino)-5-methyl-2-pyrimidinylthio]acetic acid, ethyl ester.

(4-chloro-6-[p-chlorobenzyl)methylamino]-5-methyl-2-pyrimidinylthio)acetic acid, ethyl ester.

[4-chloro-6-2,3-xylidino)-2-pyrimidinylthio]acetic acid, sodium salt, hemihydrate.

The compounds described above are routinely tested for their effect on Aβ release using in vitro tests. Routine experimentation can also be performed to determine if a composition affects the release of Aβ from at least one cell in vivo. Other suitable assays are disclosed in the Examples herein. Briefly, SM-4 cells, which are stably transfected with Swedish mutant amyloid Precursor Protein, are treated with a PPARα and/or PPARδ agonist, such as pirinixic acid, or derivative thereof. After treatment, the media is collected and assayed for Aβ-40 and/or Aβ-42. A statistically significant decrease (p<0.05) in Aβ-40 or Aβ-42 concentration in the media compared to appropriate control(s) indicates that the treatment inhibited or prevented Aβ-40 and/or Aβ-42 production and/or release from the cells. If a compound decreases Aβ-42 production and/or release by a statistically significant amount relative to control (absence of the compound or presence of vehicle) it is considered to be an Aβ-42-modulating agent according to the invention.

There is a complex relationship between AD, cholesterol homeostasis, and agents used for regulating cholesterol levels in the body. WO 00/28981 discloses the administration of an inhibitor of HMG CoA reductase (3-hydroxy-3-methylglutaryl CoA reductase) to reduce the risk of onset of Alzheimer's disease. The inhibitors used were lovastatin, pravastatin, or a combination thereof. However, a similar correlation was not seen with simvastatin. WO 00/31548 also discloses inhibitors of HMG CoA reductase, particularly statins. Interestingly, simvastatin is a suggested inhibitor, contrasting with the results disclosed in WO 00/28981, which states that the prevalence of AD in simvastatin-treated patients was not decreased.

Fassbender, K. et al., PNAS/www.pnas.org/cgi/doi/10.1073/pnas.081620098, describe the use of simvastatin to reduce levels of β-amyloid peptides Aβ-42 and Aβ-40 in vitro and in vivo, using guinea pigs. Wolozin, B. et al., Arch. Neurol. 57:1439-1443, 2000, describe the analysis of a patient population treated with HMG-CoA reductase inhibitors. The authors reported that the prevalence of AD was 60-73% lower in these patients than in patients taking other medications. In this study, a causal relationship could not be established. Jick, H. et al., The Lancet 356:1627-1631, 2000, also reviewed patient records and found that in individuals 50 years and older, statin administration was associated with a substantially lowered risk of dementia, including Alzheimer's disease and other conditions. Similarly, Acyl-CoA: cholesterol acyltransferase (ACAT) inhibitors have been used to decrease plasma cholesterol in various animal models including rats, guinea pigs and rabbits (Tanaka et al., J. Med. Chem. 41:2390-2410, 1998; Junquero et al., Biochem. Pharmacol. 61:97-108, 2001). Examples of ACAT inhibitors include but are not limited to Glibenclamide, CI-976 (PD128042), NTE-122, Fatty acid Anilides, F12511, Avasimibe, TS-962 (HL-004), N-Chlorosulfonyl isocyanate and derivatives, SR-9223i, Pyripyropenes, PD-132301, PD-132301-2, DUP-128, YM-17E, BW447A, AD 6591, CL-277,082, Melinamide, Hydroxyphenyl Urea derivatives, R-106578, Indoline derivatives with amide or urea moiety, 57-118, 58-035, CI-999, CI-1011, N-alkyl-N-[(fluorophenoxy)benzyl]-N′-arylureas and derivatives, SKF-99085, EAB309, N-alkyl-N-(heteraryl-substituted benzyl)-N′-arylureas and derivatives, F-1394, N-alkyl-N-biphenyllylmethyl-N′-aryl ureas and derivatives, CL 277,082, CL 283,546, CL 283,796, CP-113,818, CP-105,191, Polyacetylene analogs-panaxynol, panaxydol, panaxydiol and panaxytriol, T-2591, 4,4-bis(trifluoromethyl)imidazolines and derivatives, FR145237, FR186054, FR129169, Naringenin, Ulmoidol, 23-hydroxyursolic acid, 27-trans-p-coumaroyloxyursolic acid, 27-cis-p-coumaroyloxyursolic acid, Triterpenes and derivatives, N-(4,5-diphenylthiazol-2-yl)-N′-aryl or alkyl (thio)ureas and derivatives, N-(4,5-diphenylthiazol-2-yl)alkanamides and derivatives, RP73163, RP64477, Diaryl-substituted heterocyclic ureas and derivatives, Heterocyclic amides and derivatives, Cyclic sulfides derived from hetero-Diels-Alder reaction of thioaldehydes with 1,3-dienes, E5324, Tetrazole amide derivatives of (+/−)-2-dodecyl-alpha-phenyl-N-(2,4,6-trimethoxyphenyl)-2H-tetrazole-5-acetamide, Epicochlioquinone A, Acyclic(diphenylethyl) diphenylacetamides, 2-(1,3-Dioxan-2-yl)-4,5-diphenyl-1H-imidazoles and derivatives, N-(2,2-dimethyl-2,3-dihydrobenzofuran-7-yl)amide derivatives, FCE 27677, GERI-BP002-A, TMP-153, Amides of 1,2-diarylethylamines and derivatives, F-1394, N-(4-oxochroman-8-yl)amide derivatives, Terpendoles, Short chain ceramide and dihydroceramide, FY-087, 447C88, Cyclandelate, 3-quinolylurea derivatives, N-phenyl-6,11-dihydrodibenz[b,e]oxepin-11-carboxamides and related derivatives, Gypsetin, AS-183, AS-186, 2,6-disubstituted-3-imidazolylbenzopyrane derivatives, Lateritin, 2-(Alkylthio)-4,5-diphenyl-1H-imidazoles derivatives, Glisoprenins, Acaterin, U-73482, Purpactins, and Chlorpromazine.

An exemplary compound according to the invention is known as pirinixic acid. According to the examples herein, pirinixic acid induced a decrease in Aβ-42 production and/or release from SM-4 cells in a concentration-dependent manner. Although pirinixic acid is well known, the present invention is the first disclosure of its use to reduce Aβ production and/or release. Pirinixic acid has been identified as a hypolipidemic agent, and was first disclosed in U.S. Pat. No. 3,814,761 (Jun. 4, 1974), which characterized it and related compounds as anti-lipidemic agents. Although it might be tempting to view the activity of pirinixic acid on Aβ-42 production and/or release as being directly related to its hypolipidemic role, particularly in view of the clinical correlation between hypercholesterolemia and Alzheimer's disease (reviewed in Wolozin, Proc Natl Acad Sci 98:5371-5373 (2001)), in fact the mechanisms appear to be separate. Thus, a cholesterol-lowering agent is not by definition a suitable treatment for AD without further experimentation, as discussed more fully below.

Fibrates are often used as cholesterol-lowering agents but do not generally reduce Aβ-42 production and/or release. For example, SM-4 cells were treated with clofibrate and the culture media was collected in order to assay Aβ-42 levels. As shown in FIG. 2, clofibrate significantly increased Aβ-42 extracellular levels at a concentration range of 50-500 μM. Similar results were found with ETYA at 20-50 μM concentrations, as shown in FIG. 3. The fact that three PPARα agonists (all of which are cholesterol lowering agents) have disparate effects on Aβ-42 production and/or release from SM-4 cells supports the premise of the invention, which is that some PPARα agonists affect Aβ-42 production and/or release via a mechanism that is not strictly concomitant with their role as cholesterol lowering agents.

The invention therefore relates to the agents pirinixic acid and other PPARα and/or PPARδ agonists, which are capable of reducing Aβ-42 production and/or release, wherein the agent is constituted as a pharmaceutical composition, and the agent may or may not be coupled to a carrier, for example as discussed below for promoting penetration of the blood brain barrier.

3. Enhanced Penetration of Blood Brain Barrier

Compounds that may be useful in vitro or in vivo for inhibiting Aβ production and/or release from cells will be more effective in alleviating or preventing Aβ production and/or release in the brain if they can gain access to target cells in the brain. A brain cell is defined herein as any cell residing within the skull bone of the head including the spinal cord. Non-limiting examples of brain cells are neurons, glial cells (astrocytes, oligodendrocytes, microglia), cerebrovascular cells (muscle cells, endothelial cells), blood cells (red, white, platelets, etc.) and cells that comprise the meninges. However, access is restricted due to the blood brain barrier (BBB), a physical and functional blockade which separates the brain parenchyma from the systemic circulation (reviewed in Pardridge et al., J Neurovirol 5(6):556-569, 1999; Rubin and Staddon. Rev. Neurosci 22:11-28, 1999). Circulating molecules are normally able to gain access to brain cells via one of two processes: (i) lipid-mediated transport of small molecules through the BBB by free diffusion, or (ii) catalyzed transport. Thus, compounds that are useful for inhibiting Aβ production and/or release are preferably linked to agents that will facilitate penetration of the blood brain barrier. In one embodiment, the method of the present invention will employ a naturally occurring polyamine linked to a small molecule useful at inhibiting Aβ production and/or release. Natural cell metabolites that may be used as linkers, include, but are not limited to, putrescine (PUT), spermidine (SPD), spermine (SPM), or DHA. An alternative method to deliver a compound across the BBB is by intracerebroventricular pump.

The neurologic agent may also be delivered to the nasal cavity. It is preferred that the agent be delivered to the olfactory area in the upper third of the nasal cavity and particularly to the olfactory epithelium in order to promote transport of the agent into the peripheral olfactory neurons rather that the capillaries within the respiratory epithelium. In a preferred embodiment the transport of neurologic agents to the brain is accomplished by means of the nervous system instead of the circulatory system so that small molecules which inhibit Aβ production and/or release may be delivered to the appropriate areas of the brain.

It is preferable that the neurologic agent be capable of at least partially dissolving in the fluids that are secreted by the mucous membrane that surround the cilia of the olfactory receptor cells of the olfactory epithelium in order to be absorbed into the olfactory neurons. Alternatively, the agent may be combined with a carrier and/or other substances that foster dissolution of the agent within nasal releases. Potential adjuvants include GM-1, phosphatidylserine (PS), and emulsifiers such as polysorbate 80.

To further facilitate the transport of the neurologic agent into the olfactory system, the method of the present invention may combine the agent with substances that enhance the absorption of the agent through the olfactory epithelium. It is preferred that the additives promote the absorption of the agent into the peripheral olfactory receptor cells. Because of their role in odor detection, these peripheral neurons provide a direct connection between the brain and the outside environment.

The olfactory receptor cells are bipolar neurons with swellings covered by hair-like cilia which project into the nasal cavity. At the other end, axons from these cells collect into aggregates and enter the cranial cavity at the roof of the nose. It is preferred that the neurologic agent is lipophilic in order to promote absorption into the olfactory neurons and through the olfactory epithelium. Among those neurologic agents that are lipophilic are gangliosides and phosphatidylserine (PS). Alternatively, the neurologic agent may be combined with a carrier and/or other substances that enhance the absorption of the agent into the olfactory neurons. Among the supplementary substances that are preferred are lipophilic substances such as gangliosides and phosphatidylserine (PS). Uptake of non-lipophilic neurologic agents such as nerve growth factor (NGF) may be enhanced by the combination with a lipophilic substance.

In one embodiment of the method of the invention, the neurologic agent may be combined with micelles comprised of lipophilic substances. Such micelles may modify the permeability of the nasal membrane and enhance absorption of the agent. Among the lipophilic micelles that are preferred are gangliosides, particularly GM-1 ganglioside, and phosphatidylserine (PS). The neurologic agent may be combined with one or several types of micelle substances.

Once the agent has crossed the nasal epithelium, the invention further provides for transport of the neurologic agent along the olfactory neural pathway. The agent may be combined with substances that possess neurotrophic or neuritogenic properties which, in turn, may assist in transporting the agent to sites of nerve cell damage. Prophylactic therapies may apply the agent alone or in combination with a carrier, other agents, and/or other substances that may enhance the absorption of the agent into the olfactory neurons.

To deliver the agent to the olfactory neurons, the agent alone or in combination with other substances as a pharmaceutical composition may be administered to the olfactory area located in the upper third of the nasal cavity. The composition may be dispensed intranasally as a powdered or liquid nasal spray, nose drops, a gel or ointment, through a tube or catheter, by syringe, by packtail, by pledget, or by submucosal infusion.

Other modifications of the compounds described herein in order to enhance penetration of the blood brain barrier can be accomplished using methods and derivatives known in the art, including but not limited to those disclosed in the following patent publications, each of which is incorporated by reference herein:

U.S. Pat. No. 6,024,977, issued Feb. 15, 2000 to Yatvin, discloses covalent polar lipid conjugates for targeting to brain and central nervous system.

U.S. Pat. No. 5,017,566, issued May 21, 1991 to Bodor discloses β and γ cyclodextrin derivatives comprising inclusion complexes of lipoidal forms of dihydropyridine redox targeting moieties.

U.S. Pat. No. 5,023,252, issued Jun. 11, 1991 to Hseih discloses the use of pharmaceutical compositions comprising a neurologically active drug and a compound for facilitating transport of the drug across the blood-brain barrier including a macrocyclic ester, diester, amide, diamide, amidine, diamidine, thioester, dithioester, thioamide, ketone or lactone.

U.S. Pat. No. 5,024,998, issued Jun. 18, 1991 to Bodor discloses parenteral solutions of aqueous-insoluble drugs with β and γ cyclodextrin derivatives.

U.S. Pat. No. 5,039,794, issued Aug. 13, 1991 to Wier et al. discloses the use of a metastatic tumor-derived egress factor for facilitating the transport of compounds across the blood-brain barrier.

U.S. Pat. No. 5,112,863, issued May 12, 1992 to Hashimoto et al. discloses the use of N-acyl amino acid derivatives as antipsychotic drugs for delivery across the blood-brain barrier.

U.S. Pat. No. 5,124,146, issued Jun. 23, 1992 to Neuwelt discloses a method for delivery of therapeutic agents across the blood-brain barrier at sites of increase permeability associated with brain lesions.

U.S. Pat. No. 5,153,179, issued Oct. 6, 1992 to Eibl discloses acylated glycerol and derivatives for use in a medicament for improved penetration of cell membranes.

U.S. Pat. No. 5,177,064, issued Jan. 5, 1993 to Bodor discloses the use of lipoidal phosphonate derivatives of nucleoside antiviral agents for delivery across the blood-brain barrier.

U.S. Pat. No. 5,254,342, issued Oct. 19, 1993 to Shen et al. discloses receptor-mediated transcytosis of the blood-brain barrier using the transferrin receptor in combination with pharmaceutical compounds that enhance or accelerate this process.

U.S. Pat. No. 5,258,402, issued Nov. 2, 1993 to Maryanoff discloses treatment of epilepsy with imidate derivatives of anticonvulsive sulfamate.

U.S. Pat. No. 5,270,312, issued Dec. 14, 1993 to Glase et al. discloses substituted piperazines as central nervous system agents.

U.S. Pat. No. 5,284,876, issued Feb. 8, 1994 to Shashoua et al., discloses fatty acid conjugates of dopamine drugs.

U.S. Pat. No. 5,389,623, issued Feb. 14, 1995 to Bodor discloses the use of lipoidal dihydropyridine derivatives of anti-inflammatory steroids or steroid sex hormones for delivery across the blood-brain barrier.

U.S. Pat. No. 5,405,834, issued Apr. 11, 1995 to Bundgaard et al. discloses prodrug derivatives of thyrotropin releasing hormone.

U.S. Pat. No. 5,413,996, issued May 9, 1995 to Bodor discloses acyloxyalkyl phosphonate conjugates of neurologically-active drugs for anionic sequestration of such drugs in brain tissue.

U.S. Pat. No. 5,434,137, issued Jul. 18, 1995 to Black discloses methods for the selective opening of abnormal brain tissue capillaries using bradykinin infused into the carotid artery.

U.S. Pat. No. 5,442,043, issued Aug. 15, 1995 to Fukuta et al. discloses a peptide conjugate between a peptide having a biological activity and incapable of crossing the blood-brain barrier and a peptide which exhibits no biological activity and is capable of passing the blood-brain barrier by receptor-mediated endocytosis.

U.S. Pat. No. 5,466,683, issued Nov. 14, 1995 to Sterling et al. discloses water soluble analogues of an anticonvulsant for the treatment of epilepsy.

U.S. Pat. No. 5,525,727, issued Jun. 11, 1996 to Bodor discloses compositions for differential uptake and retention in brain tissue comprising a conjugate of a narcotic analgesic and agonists and antagonists thereof with a lipoidal form of dihydropyridine that forms a redox salt upon uptake across the blood-brain barrier that prevents partitioning back to the systemic circulation.

International Pat. Application Publication Number WO85/02342, published Jun. 6, 1985 for Max-Planck Institute discloses a drug composition comprising a glycerolipid or derivative thereof.

International Patent Application Publication Number WO089/11299, published Nov. 30, 1989 for State of Oregon discloses a chemical conjugate of an antibody with an enzyme which is delivered specifically to a brain lesion site for activating a separately-administered neurologically-active prodrug.

International Patent Application Publication Number WO91/04014, published Apr. 4, 1991 for Synergen, Inc. discloses methods for delivering therapeutic and diagnostic agents across the blood-brain barrier by encapsulating the drugs in liposomes targeted to brain tissue using transport-specific receptor ligands or antibodies.

International Patent Application Publication Number WO91/04745, published Apr. 18, 1991 for Athena Neurosciences, Inc. discloses transport across the blood-brain barrier using cell adhesion molecules and fragments thereof to increase the permeability of tight junctions in vascular endothelium.

International Patent Application Publication Number WO91/14438, published Oct. 3, 1991 for Columbia University discloses the use of a modified, chimeric monoclonal antibody for facilitating transport of substances across the blood-brain barrier.

International Pat. Application Publication Number WO94/01131, published Jan. 20, 1994 for Eukarion, Inc. discloses lipidized proteins, including antibodies.

International Pat. Application Publication Number WO94/03424, published Feb. 17, 1994 for Ishikira et al. discloses the use of amino acid derivatives as drug conjugates for facilitating transport across the blood-brain barrier.

International Patent Application Publication Number WO94/06450, published Mar. 31, 1994 for the University of Florida discloses conjugates of neurologically-active drugs with a dihydropyridine-type redox targeting moiety and comprising an amino acid linkage and an aliphatic residue.

International Patent Application Publication Number WO94/02178, published Feb. 3, 1994 for the U.S. Government, Department of Health and Human Services discloses antibody-targeted liposomes for delivery across the blood-brain barrier.

International Patent Application Publication Number WO95/07092, published Mar.16, 1995 for the University of Medicine and Dentistry of New Jersey discloses the use of drug-growth factor conjugates for delivering drugs across the blood-brain barrier.

International Patent Application Publication Number WO96/00537, published Jan. 11, 1996 for Southern Research Institute discloses polymeric microspheres as injectable drug-delivery vehicles for delivering bioactive agents to sites within the central nervous system.

International Patent Application Publication Number WO96/04001, published Feb. 15, 1996 for Molecular/Structural Biotechnologies, Inc. discloses omega-3-fatty acid conjugates of neurologically-active drugs for brain tissue delivery.

International Patent Application Publication Number WO96/22303, published Jul. 25, 1996 for the Commonwealth Scientific and Industrial Research Organization discloses fatty acid and glycerolipid conjugates of neurologically-active drugs for brain tissue delivery.

In general, it is well within the ordinary skill in the art to prepare an ester, amide or hydrazide derivative from the corresponding carboxylic acid and a suitable reagent. For instance, a carboxylic acid-containing compound, or a reactive equivalent thereof, may be reacted with a hydroxyl-containing compound, or a reactive equivalent thereof, so as to provide the corresponding ester. The following reference books and treatise provide exemplary reaction conditions to achieve such conversions: “Synthetic Organic Chemistry”, John Wiley & Sons, Inc., New York; S. R. Sandier et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-lnterscience, New York, 1992.

One of skill in the art can readily modify any of the agonists discussed in Sections 1 and 2 above and test them for the desired activity and ability to penetrate the blood brain barrier.

Transcytosis, including receptor-mediated transport of compositions across the blood brain barrier, is also suitable for the compounds of the invention. Transferrin receptor-mediated delivery is disclosed in U.S. Pat. Nos. 5,672,683; 5,383,988; 5,527,527; 5,977,307; and 6,015,555. Transferrin-mediated transport is also disclosed in Friden, P. M. et al., Pharmacol. Exp. Ther. 278:1491-1498, 1996; and Lee, H. J., J. Pharmacol. Exp. Ther. 292:1048-1052, 2000. EGF receptor-mediated delivery is disclosed in Deguchi, Y. et al., Bioconjug. Chem. 10:32-37, 1999, and transcytosis is described in Cerletti, A. et al., J. Drug Target. 8:435-446, 2000. The use of insulin fragments as carriers for delivery across the blood brain barrier is discussed by Fukuta, M. et al., Pharm. Res. 11:1681-1688, 1994. Delivery of compounds via a conjugate of neutral avidin and cationized human albumin is described by Kang, Y. S. et al., Pharm. Res. 1:1257-1264, 1994.

The optimal concentration of the active agent will necessarily depend upon the specific agent used, the characteristics of the patient and the nature of the disease or condition for which the treatment is to be used. The agent may be used alone or in combination with other substances as a pharmaceutical composition.

The invention is further directed to a pharmaceutical composition comprising an amount of a compound as disclosed herein, or a neurologic agent, which is effective in treating or preventing brain disorders such as Alzheimer's disease, when administered thereto, in combination with a pharmaceutically acceptable vehicle such as a liquid or powdered carrier and/or various optional adjuvants.

In one embodiment, the invention provides method of treatment comprising modulating the production and/or release of β-amyloid in a non-human mammal in need of said treatment. In another embodiment, the invention provides method of treatment comprising modulating the production and/or release of β-amyloid in a human in need of said treatment. Whether the treating is to human or non-human mammals, the inventive method comprises administering to said subject a compound or composition as described herein, and particularly a compound selected from compounds of the formulae

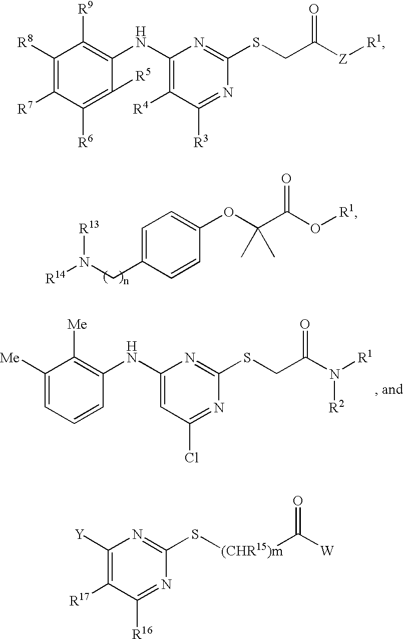

each as defined herein, including various embodiments thereof. In various embodiments, in these compounds, —Z—R¹ represents —OH, —O—R¹ represents —OH, —N(R¹(R²) represents —NH₂, and W represents —OH, so that the above compounds are either carboxylic acids or primary amides. In various other embodiments, —Z—R¹ represents —O⁻, —O—R¹ represents —O⁻, and W represents —O⁻, so that the above compounds are carboxylates, which are in association with a counterion selected from metal cations and ammonium cations. In a preferred embodiment, the groups R¹ or W impart enhanced penetration of the blood brain barrier to the compound, relative to the otherwise identical compound having R¹ or W as H or OH so as to provide the carboxylic acid.

These compounds may also be used to modulate the production and/or release of β-amyloid in a cell, by treating said cell with an effective amount of the compound or a composition containing the compound. As used herein, the term “a” refers to one or more, so that, for example, “a compound” refers to one or more compounds.

In one embodiment, the compound or composition as described herein is used to treat a human, wherein said human is afflicted with Alzheimer's disease. In another embodiment, said human being treated has a genetic predisposition or environment exposure that increases the likelihood that said person will develop Alzheimer's disease. For example, said human has suffered a head injury and is treated with a compound or composition as described herein. In one embodiment, said human exhibits minimal cognitive impairment suggestive of early stage Alzheimer's disease. In another embodiment, said human has suffered a head injury and is treated with a compound or composition as described herein.

The carrier of the composition may be any material that is otherwise pharmaceutically acceptable and compatible with the active ingredients of the composition. Where the carrier is a liquid, it is preferred that the carrier is hypotonic or isotonic with nasal fluids and within the range of pH 4.5-7.5. Where the carrier is in powdered form, it is preferred that the carrier is also within an acceptable non-toxic pH range.

Among the optional substances that may be combined with the neurologic agent in the pharmaceutical composition are lipophilic substances that may enhance absorption of the agent across the nasal membrane and delivery to the brain by means of the olfactory neural pathway. The neurologic agent may be mixed with a lipophilic adjuvant alone or in combination with a carrier. Among the preferred lipophilic substances are gangliosides and phosphatidylserine (PS). One or several lipophilic adjuvants may be combined with the agent. It is preferred that the lipophilic adjuvant be added as micelles.

The pharmaceutical composition may be formulated as a powder, granules, solution, ointment, cream, aerosol, powder, or drops. The solution may be sterile, isotonic or hypotonic, and otherwise suitable for administration by injection or other means. In addition to the neurologic agent, the solution may contain appropriate adjuvants, buffers, preservatives and salts. The powder or granular forms of the pharmaceutical composition may be combined with a solution and with diluting, dispersing and/or surface active agents. Solutions such as nose drops may contain antioxidants, buffers, and the like.

Routine experimentation can be performed to determine in vitro if a composition will be capable of penetrating the blood brain barrier in vivo. For example, using monolayer culture models, substances can be added to one side of the culture and test performed to see if the compound can be detected on the other side of the culture.

An in vitro model of the blood brain barrier is described in Gaillard et al., Eur. Jour. Pharm. Sci. 12:215-222, 2001. In this model, brain capillary endothelial cells are co-cultured with astrocytes. A separate system utilizes brain microvessel endothelial cells, as described by Franke et al., Brain Res. Protocols 5:248-256, 2000. According to the authors this model system is in use for preclinical research on drugs for treating the central nervous system, and the publication provides detailed steps for measuring permeation, for example by using radiolabeled drug. Another model, consisting of an immortalized cell line of rat capillary cerebral endothelial cells, is described in Martel et al., Naunyn-Schmiedeberg's Archives of Pharmacology, Sep. 5, 2000. Application of blood brain barrier principles to specific classes of drugs is discussed in Pardidge, W. M., Jour. Neurochem. 70:1781-1792, 1998.

Terasaki et al., Biol. Pharm. Bull. 24:111-118 (2001) describe conditionally immortalized cell lines as models for the blood brain barrier, particularly for drug transport to the brain.

In vivo models may also be used. The agent is radiolabeled or fluorescently labeled and administered peripherally by intravenous injection (Pan, W., et al., Neuropharmacol. 37:1553-1561, 1998), orally (Shulkin, B. L. et al., J. Neurochem. 64:1252-1257, 1995) or nasally (Thorne, R. G. et al., Brain Res. 692:278-282, 1995) and the concentration of the agent in the blood as compared to the brain is monitored. Similar models are well known in the art.

In addition to PPARα agonists, PPARδ agonists may also be suitable for use according to the invention. PPARδ agonists and activators are described in Willson, T. M. et al., Jour. Med. Chem. 43:527-550, 2000. The PPARδ receptor is believed to play a role in lipid homeostasis, including cholesterol homeostasis. For example, Oliver, W. R. et al. (Proc. Nat'l. Acad. Sci. 98:5306-5311, 2001) showed that administration of the PPARδ agonist GW501516 to obese monkeys resulted in an increase in serum HDL cholesterol. In separate experiments, at a concentration of about 50 μM, pirinixic acid was found to be an effective agonist of PPARδ as measured by alteration in cholesterol efflux (Oliver, W. R. et al. Proc. Nat'l. Acad. Sci. 98:5306-5311, 2001). This is comparable to the concentrations used in the present invention using pirinixic acid as an agonist of PPARα. Other PPAR agonists suitable for use include a ureido-thioisobutyric acid (GW 9578) and derivatives, as described in Brown, P. B. et al. (J. Med. Chem. 43:3785-3788, 1999).

An exemplary and preferred compound is a derivative of pirinixic acid, wherein the molecule has been esterified to facilitate penetration of the blood brain barrier:

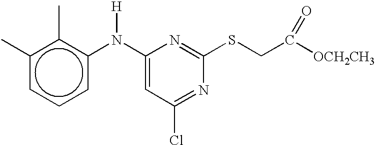

Another preferred compound consists of pirinixic acid conjugated to DHA, which also facilitates penetration of the blood brain barrier:

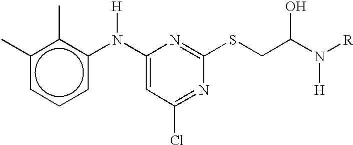

where R is derived from DHA.

Additional derivatives of pirinixic acid and other compounds of the invention can be prepared in order to facilitate their penetration of the blood brain barrier, using methods known in the art. For example, U.S. Pat. No. 6,024,977 discloses neurologically active compounds with covalent polar lipid conjugates. The polar lipid carrier includes sphingosinse, ceramide, phosphatidyl choline, phosphatidyl glycerol, phosphatidyl ethanolamine, phosphatidyl inositol, phosphatidyl serine, cardiolipin, phosphatidic acid, sphingomyelin, and other sphingolipids. Optionally, a spacer may be placed between the lipid moiety and the biologically active component, and the spacer may comprise a polypeptide of, for example, 2 to 25 amino acids. In another example, U.S. Pat. No. 6,197,764 discloses conjugates of a fatty acid molecule and a bioactive compound; a preferred fatty acid is docosahexaenoic acid (DHA). In a further example, U.S. Pat. No. 5,994,392 discloses prodrugs that pass through the blood brain barrier, comprising a fatty acid carrier of 16 to 26 carbon atoms, wherein the fatty acid carrier is a partially-saturated straight chain molecule. The covalent bond between the drug and carrier is preferably an amide bond.

The invention is also described with reference to the following examples, which are not intended to be limiting. All patents and publications referenced above and in the Examples are incorporated by reference herein.

### EXAMPLES

### Example 1

### Effect of Pirinixic Acid Treatment on β-Amyloid Production and/or Release from Cells

Cell Lines and Pharmacological Treatments. 293 EBNA cells (InVitrogen, Carlsbad, Calif.) stably transfected with Swedish mutant Amyloid Precursor Protein −695 (SM4 cells) were routinely maintained in DMEM supplemented with sodium pyruvate (1 mM) and 10% fetal bovine serum. Cells were seeded into poly-D-Lysine (SIGMA) coated 6-well plates at a density of 5-7×10⁵ cells per well. Subsequently, the cells were rinsed in 1 ml of PBS and treated with 10-500 μM of pirinixic acid in serum-free/phenol red-free DMEM for 16 hours.

Aβ Detection and Standardization. After the pharmacological treatment, the exposure media was collected and supplemented with 10% sample treatment buffer (40 mM sodium phosphate (pH 7.4), 40 mM triethanolamine, 0.1% Triton X-100, 200 mM NaCl, 2 mM EGTA, 0.1% Sodium azide), and assayed for either Aβ-40 or Aβ-42 by a colorimetric ELISA as per the manufacturer's protocol (Biosource International Inc, California). The cells were lysed in 0.1% Triton X-100 in PBS supplemented with 5 μM propridium iodide (Molecular Probes, Eugene, Oreg.) and incubated at 37° C. for 30 minutes prior to measuring fluorescence. Aβ-40 and Aβ-42 were standardized against propridium iodide fluorescence as a measure of total cell number.

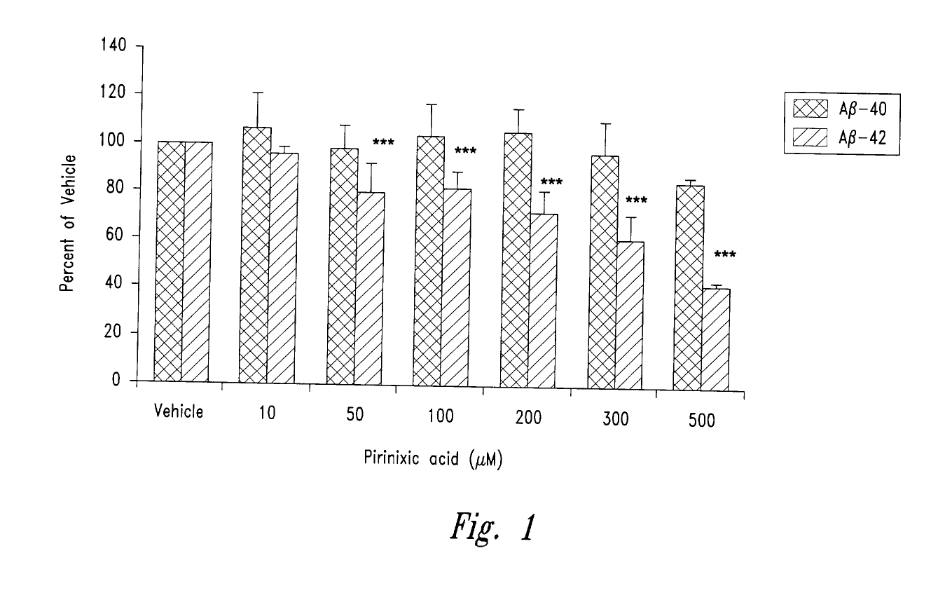

The PPARα and/or PPARδ agonist, pirinixic acid induced a significant decrease in Aβ-42 production and/or release from SM-4 cells after 16 hrs. Concentrations as low as 50 μM induced a 15% decrease (p<0.001) in Aβ-42. At 500 μM a 60% decrease in Aβ-42 was observed (FIG. 1). Interestingly, the pirinixic acid mediated decrease in Aβ production and/or release was selective since there was no significant change in Aβ-40 production and/or release.

### Example 2

### Screening Agents for Ability to Decrease β-Amyloid Production and/or Release from Cells

293 EBNA cells stably transfected with Swedish mutant Amyloid Precursor Protein −695 are maintained in DMEM supplemented with sodium pyruvate (1 mM) and 10% fetal bovine serum. Cells are seeded into Poly-D-Lysine coated 6-well plates at a density of 5-7×10⁵ cells per well. Subsequently, the cells are rinsed in 1 ml of PBS and treated with 10-500 μM of a PPARα or a PPARδ agonist in serum-free/phenol red-free DMEM for 16 hours.

After the pharmacological treatment, the exposure media is collected and supplemented with 10% sample treatment buffer (40 mM sodium phosphate (pH 7.4), 40 mM triethanolamine, 0.1% Triton X-100, 200 mM NaCl, 2 mM EGTA, 0.1% Sodium azide), and assayed for either Aβ-40 or Aβ-42 by a colorimetric ELISA as per the manufacturer's protocol (Biosource International, Inc., California). The cells are lysed in 0.1% Triton X-100 in PBS supplemented with 5 μM propridium iodide (Molecular Probes, Eugene, Oreg.) and incubated at 37° C. for 30 minutes prior to measuring fluorescence. Secreted Aβ-40 and Aβ-42 are standardized against propridium iodide fluorescence as a measure of total cell number.

### Example 3

### Screening Agents for Ability to Penetrate Blood Brain Barrier

Using an in vitro model such as that disclosed in Franke, H. et al., Brain Res. Prot. 5:248-256, 2000, or an in vivo model such as those described by Shulkin, B. L. et al., J. Neurochem. 64:1252-1257, 1995; Thorne, R. G. et al., Brain Res. 692:278-282, 1995; Pan, W., et al., Neuropharmacol. 37:1553-1561, 1998, pharmaceutical agents of the invention can be routinely tested for their ability to penetrate the blood brain barrier. The in vitro model uses a PBEC (porcine brain microvessel endothelial cell) monolayer which is arranged so that the ability of substances to pass from a donor compartment to an acceptor compartment can be measured. This model reflects the in vivo situation wherein substances reach the brain compartment from a brain microvessel. Permeation properties of an agent of the invention are measured by radiolabeling the agent, for example with ³H, and adding it to the donor compartment. Samples are collected from the donor and acceptor compartments at routine intervals and permeability is calculated as described in Franke, H. et al., (2000).

The in vivo models measure the brain influx index or the measure of the passage of a substance through the blood brain barrier. The agent is radiolabeled or fluorescently labeled and administered peripherally by intravenous injection (Pan, W., et al., Neuropharmacol. 37:1553-1561, 1998), orally (Shulkin, B. L. et al., J. Neurochem. 64:1252-1257, 1995) or nasally (Thorne, R. G. et al., Brain Res. 692:278-282, 1995) and the concentration of the agent in the blood as compared to the brain is monitored.

### Example 4

### Effect of Pirinixic Acid Treatment on Production of Amyloid Precursor Protein and Proteolytic Fragments Thereof

Cell Lines and Pharmacological Treatments. SM4 cells were routinely maintained, seeded into Poly-D-Lysine (SIGMA) coated 6-well plates, rinsed in PBS, and treated with 50-500 μM of pirinixic acid in serum free/phenol red free DMEM for 16 hours as described in Example 1.

Detection of Amyloid Precursor Protein and its Proteolytic Fragments. After the pharmacological treatment, the conditioned media was harvested and the cellular lysate was collected in 100 μl of cold SAPK lysis buffer (0.01% Nonidet P-40, 20 mM MOPS 5 mM EDTA and 75 mM β-glycerol phosphate, protease inhibitor cocktail (Boehringer Mannheim, Laval, QC)) and sonicated on ice for 8 seconds using a probe sonicator. From each sample, total protein concentration was determined using the bicinchonic acid assay (Pierce, Rockford, Ill., USA). Cellular APP and secreted APP_{sα} levels were quantitated by 10% Tris-Glyine SDS-PAGE Western blot analysis using an anti-APP N-terminal antibody (22C11, Boehringer Mannheim, Laval, QC) (Mills et al., 1997; Connop et al., 1999) and monoclonal 6E10 (Senetek Research, Maryland Heights, Mo., USA), respectively. C99 was quantitated from the cellular lysate by 16.5% Tris-Tricine SDS-PAGE Western blot analysis using monoclonal antibody 6E10 (Senetek Research, Maryland Heights, Mo., USA). Immunoreactive bands were visualized using ECL detection (Amersham, Oakville, ON) and analyzed by standard densitometric techniques.

Statistical Analysis. Statistical significance was determined using an ANOVA with Tukey's post hoc analysis. Data are expressed as mean±SD with *p<0.05 and **p<0.01 and n=4.

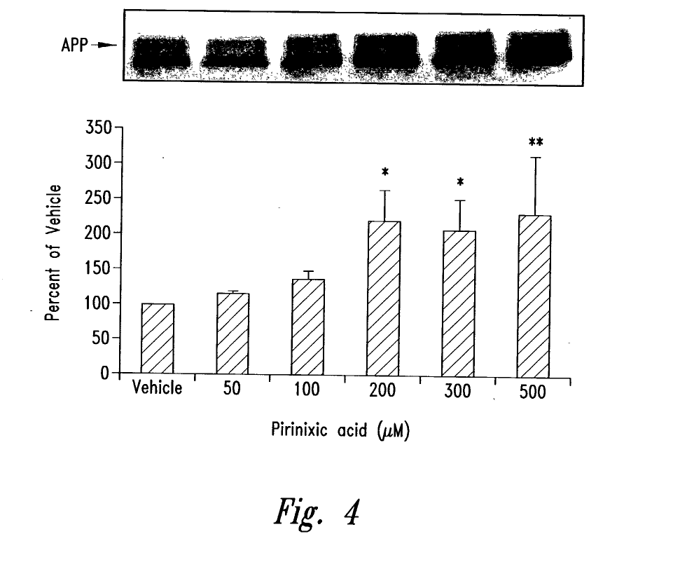

Result. FIG. 4 shows the effect of PPARα and/or PPARδ agonist pirinixic acid on cellular APP levels from SM-4 cells quantitated by Western blot analysis. A representative micrograph of the C99 Western blot data is depicted above the corresponding densitometric values. Data are expressed as mean±SD with n=4 and statistical significance determined by ANOVA with Tukey's post hoc test at *p<0.05 and **p<0.01.

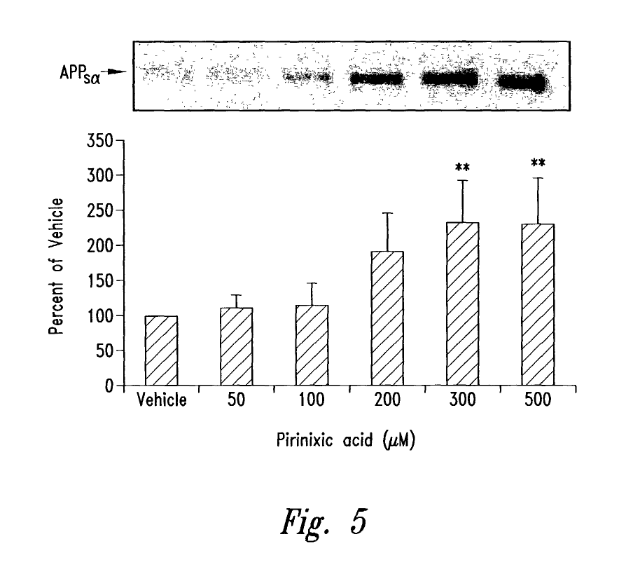

FIG. 5 shows the effect of PPARα and/or PPARδ agonist pirinixic acid on APP_{sα} release from SM-4 cells quantitated by Western blot analysis. A representative micrograph of the C99 Western blot data is depicted above the corresponding densitometric values. Data are expressed as mean±SD with n=4 and statistical significance determined by ANOVA with Tukey's post hoc test at **p<0.01.

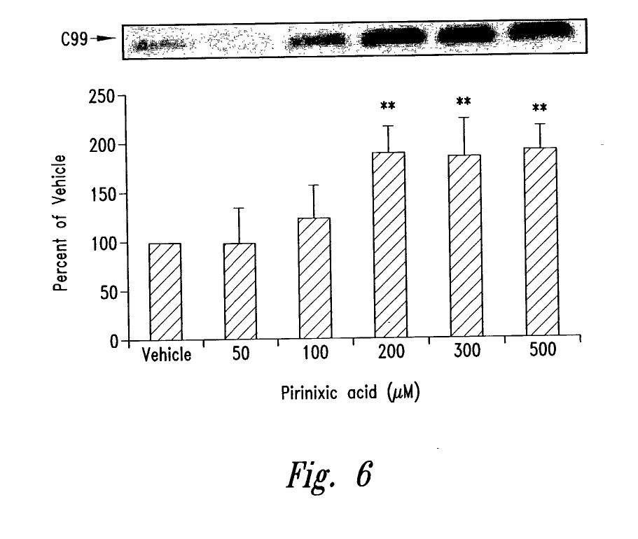

FIG. 6 shows the effect of PPARα and/or PPARδ agonist pirinixic acid on C99 levels from SM-4 cells quantitated by Western blot analysis. A representative micrograph of the C99 Western blot data is depicted above the corresponding densitometric values. Data are expressed as mean±SD with n=4 and statistical significance determined by ANOVA with Tukey's post hoc test at **p<0.01.

### Example 5

### The Effect of Pirinixic Acid on Aβ-40/42 Production and/or Secretion from Human Neuroblastoma Cells

Cell Lines and Pharmacological Treatment

Human neuroblastoma cells (hDAT; SK-N-MC stably overexpression human dopamine transporter) were routinely maintained in DMEM supplemented with sodium pyruvate (1 mM) and 10% fetal bovine serum. Cells were seeded into 6-well plates at a density of 2.5×10⁵ cells per well and transiently transfected with APPsw (Swedish mutant amyloid precursor protein-695) using lipofectamine (Life Technologies, Rockville, Md.) as per the manufacturer's suggested protocol. Subsequently, 48 hours post-transfection the cells were rinsed with PBS and treated with vehicle (0.1% DMSO) or 100-200 μM pirinixic acid in serum free/phenol free DMEM for 24 hours.

Aβ Detection and Standardization

After the pharmacological treatment, the exposure media was collected and supplemented with 10% sample treatment buffer (40 mM sodium phosphate (pH 7.4), 40 mM triethanolamine, 0.1% Triton X-100, 200 mM NaCl, 2 mM EGTA, 0.1% Sodium azide), and assayed for either Aβ-40 or Aβ-42 by a colorimetric ELISA as per the manufacturer's protocol (Biosource International Inc, California). The cells were lysed in 0.1% Triton X-100 in PBS supplemented with 5 μM Propridium Iodide (Molecular probes, Eugene, Oreg.) and incubated at 37° C. for 30 minutes prior to measuring fluorescence. Secreted Aβ-40 and Aβ-42 levels were standardized against propridium iodide fluorescence as a measure of total cell number.

Statistical Analysis

Data are expressed as a percent of control and represent the mean±SD with n=11 and statistical significance determined by ANOVA with a Tukey's post hoc test at ***p<0.001.

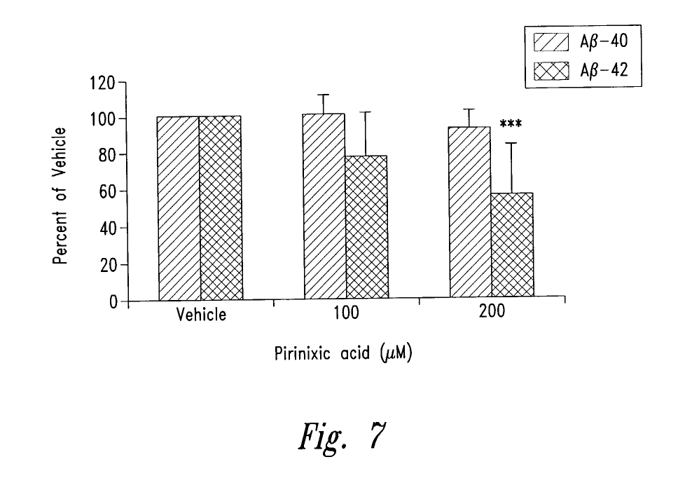

FIG. 7 demonstrates the effects of PPARα and/or PPARδ agonist pirinixic acid on Aβ-40/42 from human neuroblastoma cells transiently transfected with APPsw. A concentration of 200 μM pirinixic acid selectively decreases Aβ-42 by 40% (p<0.001, n=11) without altering Aβ-40.

### Example 6

### The Effect of Pirinixic Acid on AβTotal and Aβ-42 Production and/or Secretion from Primary Murine Cortical Neurons

Semliki Forest Virus (SFV) stocks

The cDNA coding for human APP695 was cloned in the SmaI site of pSFV-1 as described previously (Simons et al., J. Neurosci. 16:899-908, 1996; Tienari et al., Embo. J. 15:5218-29, 1996). PSFV-1/huAPP695 constructs were linearized with SpeI and run-off transcription using SP6 polymerase was performed to produce mRNA. The transcribed mix of APP and pSFV-helper were cotransfected into BHK cells by electroporation to yield recombinant SFV (Olkkonen et al., J. Neurosci. Res. 35:445-51, 1993). BHK cells were grown in DMEM/F12 supplemented with 5% fetal calf serum, 2 mM L-glutamine, 100 U/ml penicillin, and 100 mg/ml streptomycin. Twenty-four hours after transfection, the culture supernatant containing infective recombinant SFV was collected. Aliquots were snap-frozen in liquid nitrogen and stored at −70° C. until use.

Neuronal Culture

All experiments were conducted on murine primary cortical neurons derived from E14 embryos according to established procedures (Annaert et al., J. Cell Biol. 147:277-294, 1999; Cupers et al., J. Cell Biol. 154:731-40, 2001; De Strooper et al., Nature 391:387-90, 1998). Briefly, cortices of 14-day-old murine embryos were dissected, transferred to Hanks' Balanced Salt Solution (HBSS, Gibco BRL, Rockville, Md.) and trypsinized for 15 minutes at 37° C. Dissociated cell suspensions were routinely plated on poly-L-lysine (l mg/ml, Sigma, St. Louis, Mo.) coated dishes (Nunc, Naperville, Ill.) in Minimal Essential Medium (MEM; Gibco BRL) supplemented with 10% horse serum and transferred to a CO₂ incubator. After 3 hours, the culture medium was replaced by serum-free neurobasal medium with B27 supplement (Gibco BRL). After 24 hours, cytosine arabinoside (5 μM) was added to each dish to prevent nonneuronal (glial) cell proliferation. Three to four days post-plating, mixed cortical neuron cultures were used for drug testing.

Semliki Forest Virus Infection

Cortical neurons were incubated with increasing concentrations of pirinixic acid (stock solution 400 mM in DMSO). First, a concentrated dilution series was prepared in DMSO comprising 4, 20, 40 and 200 mM compound. From each of these solutions, 2.5 μl was added to the neuronal cultures in 2 ml of neurobasal medium (dilution 1/800) resulting in 5, 25, 50 and 250 μM final concentrations. As a control, 2.5 μl of DMSO was added to one dish.

After an overnight (16 hours) incubation at 37° C., the medium was replaced by 1.2 ml neurobasal medium and cultures were transduced by adding recombinant pSFV-humAPP695wt (dilution 1/10) for 1 hour to allow viral entry. Following a 2-hour incubation in the absence of virus, cultures were metabolically labeled using methionine-free neurobasal medium containing 100 μCi [35S]-methionine (ICN). After 4 hours at 37° C., the conditioned medium and the cell extracts were collected and centrifuged (14,000 rpm, 15 min).

Detection of Aβtotal and APP from Conditioned Media

The cleared fractions were subject to immunoprecipitation with different antibodies on protein G-Sepharose (Pharmacia). Pab B11, recognizing the last 20 amino acids of APP (De Strooper et al., Embo J. 14:4932-8, 1995), was added to the cell extracts to immunoprecipitate APP. Aβ total was examined from the cleared conditioned media by immunoprecipitation using pab B7, directed against the first 17 amino acids of Aβ (De Strooper et al., Embo. J. 14:4932-8, 1995). After overnight rotation, the immunoprecipitates were washed 5 times in extraction buffer and once in TBS. The bound material was denatured in sample buffer and subject to gel electrophoresis on precast 10% or 4-12% Nupage gels for APP and Aβ total, respectively. Densitometric analysis was conducted using a Phosphoimager (Molecular Dynamics) and ImagQuant 5.0. Aβ total levels were normalized to APP levels to control for plate to plate variation.

Quantification of Aβ-42 by ELISA

The levels of the longer Aβ-42 peptide were quantified in both the conditioned media and cell extracts using a sandwich ELISA test ((De Strooper et al., Nature 391:387-90, 1998) and Innogenetics, Ghent, Belgium) and according to the manufacturer's instructions (see also Vanderstichele et al., Amyloid. 7:245-58, 2000). In summary, 800 μl of conditioned medium or cell extract was lyophilized (Savant Speedvac concentrator), dried pellets were dissolved in 400 μl of sample diluent and applied on a 96-well ELISA plate precoated with the capturing anti-Aβ-42 mab 21F12. This antibody only recognizes the final two amino acids of the Aβ-42 sequence. After washing, the wells were incubated with biotin-labeled mAb 3D6 directed against the first 7 amino acids of Aβ, followed by streptavidine-HRP. Finally HRP substrate was added and the colorimetric reaction was quantitated spectrophotometrically using a Victor 2 (Wallac) equipped with a 450 nm filter. For each experiment a duplicate standard curve for Aβ-42 was included. The Aβ-42 concentrations in the samples were finally calculated based on the Aβ-42 standards nonlinear regression equation and using Mathematica 4.1 software package (Wolfram Research, Champaign, Ill.).

Statistical Analysis Data are expressed as a percent of control and represent the mean±SD with n=6 and statistical significance determined by ANOVA with a Tukey's post hoc test at **p<0.01, ***p<0.001.

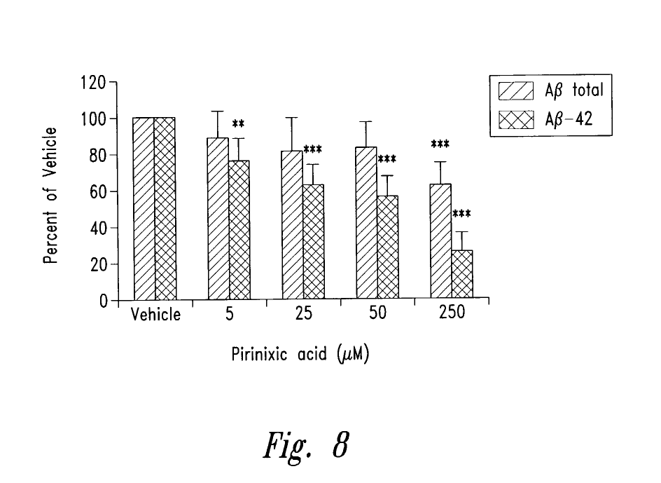

FIG. 8 demonstrates the effects of PPARα and/or PPARδ agonist pirinixic acid on Aβtotal and Aβ-42 levels from primary murine cortical neurons infected with APP695. A concentration dependant decrease in Aβ-42 was observed. A 20% decrease in Aβ-42 was observed at 5 μM pirinixic acid (p<0.01, n=6). In contrast, no significant effect on Aβtotal was observed until cells were treated with 250 μM pirinixic acid. This data demonstrates a selective decrease in Aβ-42 at 5-50 μM pirinixic acid without altering Aβtotal.

All of the above U.S. patents, U.S. patent application publications, U.S. patent applications, foreign patents, foreign patent applications and non-patent publications referred to in this specification and/or listed in the Application Data Sheet, including but not limited to U.S. patent application Ser. No. 60/297,845 filed Jun. 12, 2001 and U.S. patent application Ser. No. 60/309,257 filed Jul. 31, 2001, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound of the formula 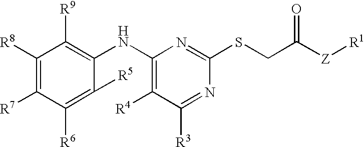
wherein, independently at each occurrence,
R¹ is an organic moiety having at least 4 carbons;
Z is selected from —O—, —NH—NH—, and —N(R²)—;
R² is selected from hydrogen and C₁-C₃₀ organic moieties with the proviso that R¹ and R² can join together with the nitrogen to which they are both attached and form a heterocyclic moiety;
R³ and R⁴ are each independently selected from the group consisting of hydrogen, halogen, lower alkyl and lower alkoxy radicals;
R⁵, R⁶, R⁷, R⁸ and R⁹ are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R¹⁰—N═N—O—R¹¹, —OR¹², —C(O)OR¹², —N(R¹²)₂, —C(O)N(R¹²)₂, —N(R¹²)C(O)OR¹¹, heterocyclyl and heterocyclylalkyl;
R¹⁰ is a bond or a straight or branched alkylene or alkenylene chain;
R¹¹ is hydrogen, alkyl or aralkyl; and
R¹² is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl;
with the proviso that Z is not NR² when R³ is Cl, R⁴ is H, R⁵ is H, R⁶ is H, R⁷ is H, R⁸ is methyl and R⁹ is methyl.

2. A compound of claim 1 wherein Z is —O— and R¹ is an organic group having less than 30 carbons and a formula weight of less than 1,000.

3. A compound of claim 1 wherein Z is —N(H)— and R¹ is an organic group having less than 30 carbons and a formula weight of less than 1,000.

4. A compound of claim 1 wherein Z is —N(R²)— and R¹ is an organic group having less than 30 carbons and a formula weight of less than 1,000.

5. A compound of claim 1 wherein R¹ is selected from the group consisting of alkyl, alkenyl, aryl, aralkyl, aralkenyl, cycloalkyl, cycloalkylalkyl, cycloalkylalkenyl, halogen, haloalkyl, haloalkenyl, cyano, nitro, —R¹⁰—N═N—O—R¹¹, —OR¹², —C(O)OR₁₂, —N(R¹²)₂, —C(O)N(R¹²)₂, —N(R¹²)C(O)OR¹¹, heterocyclyl and heterocyclylalkyl.

6. A compound of claim 1 wherein R¹ is a straight-chained hydrocarbon moiety containing between 16 and 26 carbon atoms, wherein the moiety is selected from the group consisting of C16:0; C16:1; C16:2; C20:1; C20:2; C20:3; C20:4; C22:4; C22:5; C22:6 and C24:4.

7. A compound of claim 1 wherein R¹ is a fragment of insulin wherein said insulin fragment binds to an insulin receptor.

8. A compound of claim 7 wherein said fragment of insulin consists of:
(a) a peptide chain having 14 to 21 amino acid residues from the N-terminus of insulin chain A; and
(b) another peptide chain having 16 to 22 amino acid residues from the N-terminus of insulin chain B.

9. A compound of claim 1 wherein R¹ is a protein that binds to a transferrin receptor.

10. A compound of claim 1 wherein R¹ is an antibody or a fragment thereof capable of binding to a ligand in the brain.

11. A compound of claim 10 wherein said antibody is a monoclonal antibody.

12. A compound of claim 1 wherein R¹ is a growth factor.

13. A compound of claim 12 wherein said growth factor is EGF.

14. A compound of claim 1 wherein each of R⁵, R⁶, R⁷, R⁸ and R⁹ is independently selected from the group consisting of hydrogen, halogen, lower alkyl and lower alkoxy radicals.

15. A compound of claim 1 having enhanced penetration of the blood brain barrier relative to the corresponding compound wherein R¹ is hydrogen when Z is —O—, and both R¹ and R² are hydrogen when Z is —N(R²)—.

16. A composition comprising a compound of any one of claims 1-15 and a pharmaceutically acceptable carrier, diluent or excipient.

17. A compound of the formula 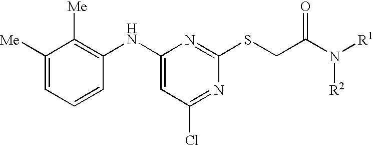
wherein,
R¹ is a hydrophobic moiety selected from non-aromatic organic moieties having at least 10 carbon atoms and aromatic moieties having at least 6 carbons, and R² is hydrogen; or
each of R¹ and R² are selected from hydrophobic organic moieties having at least one carbon atom, with the proviso that R¹ and R² in total have at least six carbon atoms, and with the further proviso that R¹ and R² can join together with the nitrogen to which they are both bonded and form a heterocyclic moiety.

18. A composition comprising a compound of claim 17 and a pharmaceutically acceptable carrier, diluent or excipient.

19. A compound that (1) is a PPARα agonist and/or a PPARδ agonist, and (2) regulates the production and/or release of β-amyloid in cells.

20. A compound of claim 19 having the formula 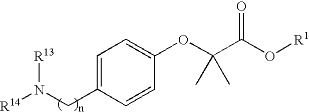
wherein,
R¹ is an organic moiety having at least 4 carbons;
R¹³and R¹⁴are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R¹⁰—N═N—O—R¹¹, —OR¹², —C(O)OR¹², —N(R¹²)₂, —C(O)N(R¹²)₂, —N(R¹²)C(O)OR¹¹, heterocyclyl and heterocyclylalkyl;
R¹⁰ is a bond or a straight or branched alkylene or alkenylene chain;
R¹¹ is hydrogen, alkyl or aralkyl; and
R¹² is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl; and
n is 1, 2 or 3.

21. A compound of claim 20 wherein R¹³ is selected from 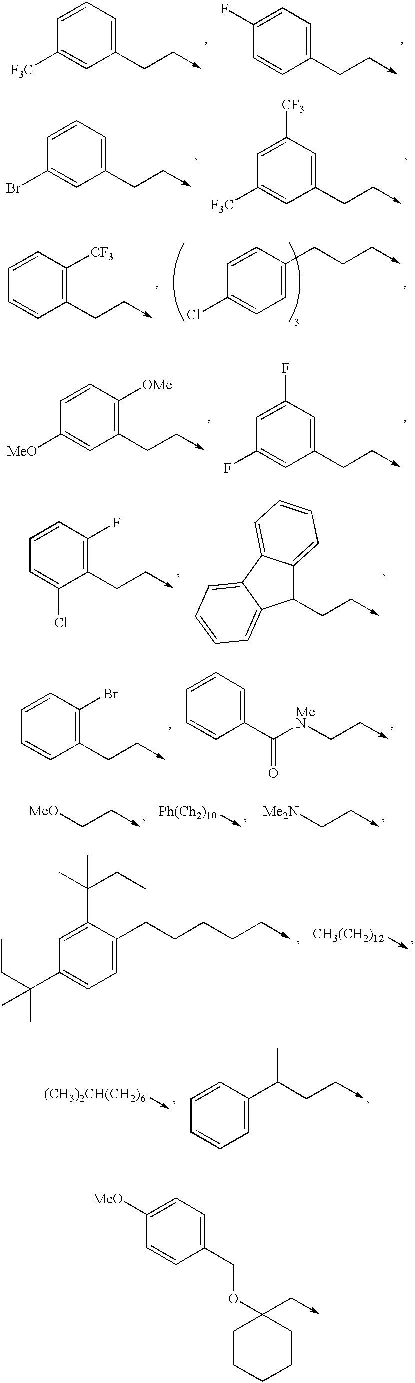
and R¹⁴ is selected from

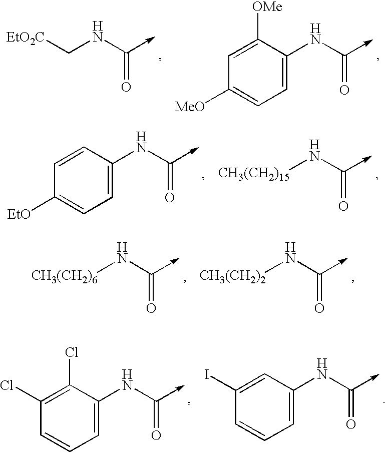

22. A compound of claim 20 having the formula 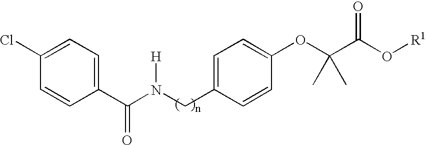
wherein n is 2.

23. A compound of claim 20 having the formula 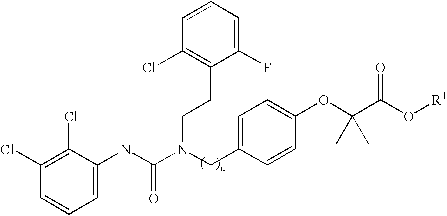
wherein n is 3.

24. A compound of claim 20 wherein R¹ is an organic group having less than 30 carbons and a formula weight of less than 1,000.

25. A compound of claim 20 wherein R¹ is selected from the group consisting of alkyl, alkenyl, aryl, aralkyl, aralkenyl, cycloalkyl, cycloalkylalkyl, cycloalkylalkenyl, halogen, haloalkyl, haloalkenyl, cyano, nitro, —R¹⁰⁻N═N—O—R¹¹, —OR¹², —C(O)OR¹², —N(R¹²)₂, —C(O)N(R¹²)₂, —N(R¹²)C(O)OR¹¹, heterocyclyl and heterocyclylalkyl.

26. A compound of claim 20 wherein R¹ is a straight-chained hydrocarbon moiety containing between 16 and 26 carbon atoms, wherein the moiety is selected from the group consisting of C16:0; C16:1; C16:2; C20:1; C20:2; C20:3; C20:4; C22:4; C22:5; C22:6 and C24:4.

27. A compound of claim 20 wherein R¹ is a fragment of insulin wherein said insulin fragment binds to an insulin receptor.

28. A compound of claim 27 wherein said fragment of insulin consists of:
(a) a peptide chain having 14 to 21 amino acid residues from the N-terminus of insulin chain A; and
(b) another peptide chain having 16 to 22 amino acid residues from the N-terminus of insulin chain B.

29. A compound of claim 20 wherein R¹ is a protein that binds to a transferrin receptor.

30. A compound of claim 20 wherein R¹ is an antibody or a fragment thereof capable of binding to a ligand in the brain.

31. A compound of claim 30 wherein said antibody is a monoclonal antibody.

32. A compound of claim 20 wherein R¹ is a growth factor.

33. A compound of claim 32 wherein said growth factor is EGF.

34. A compound of claim 20 having enhanced penetration of the blood brain barrier relative to the corresponding compound wherein R¹ is hydrogen.

35. A composition comprising a compound of any one of claims 19-34 and a pharmaceutically acceptable carrier, diluent or excipient.

36. A method for modulating the production and/or release of β-amyloid in a cell, comprising treating said cell with a compound according to any one of claims 1-15, 17 or 19-34, or a composition according to any one of claims 16, 18 and 35.

37. A method for modulating the production and/or release of β-amyloid in a cell, comprising treating said cell with a compound of the formula 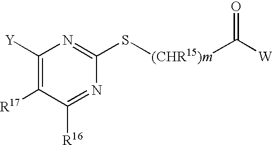
wherein, independently at each occurrence,
R¹⁵ and R¹⁷ are each independently selected from the group consisting of hydrogen and lower alkyl radicals;
R¹⁶ is selected from the group consisting of hydrogen, halogen and lower alkoxy radicals;
W is selected from the group consisting of hydroxy, lower alkoxy, —OM and —(NH)ₚNH₂ radicals, wherein p is 0 or 1, and M is an alkali metal cation, an alkaline earth metal cation or the ammonium ion;
m is 0, 1, 2 or 3;
Y is selected from the group consisting of an aryl radical of 6 to 10 carbon atoms; 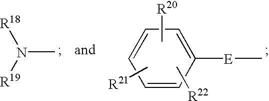
wherein
R¹⁸ is hydrogen or lower alkyl radical;
R¹⁹ is hydrogen, H₂N—, 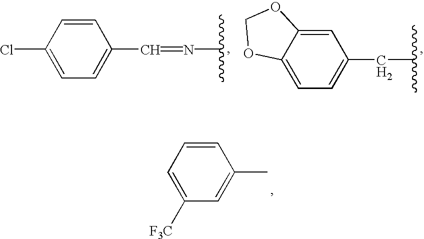
phenyl, (lower)alkoxyphenyl, or di(lower)alkoxy-phenyl, providing that when R¹⁸ is hydrogen and R¹⁹ is hydrogen, phenyl, (lower)alkoxyphenyl or di(lower)alkoxyphenyl, R¹⁶ is halo or lower alkoxy,
R²⁰ is selected from the group consisting of a lower alkyl radical, a halo radical, an aryl radical of 6 to 10 carbon atoms and a haloaryl radical of 6 to 10 carbon atoms,
R²¹ is selected from the group consisting of hydrogen, lower alkyl, lower alkoxy and halo radicals,
R²² is selected from the group consisting of hydrogen and lower alkyl radicals, and
E is selected from the group consisting of 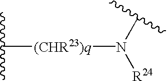
and 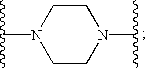
wherein
R²³ is hydrogen or lower alkyl
R²⁴ is hydrogen or lower alkyl, and
q is an integer from 0 to 3.

38. The method of claim 36 or 37 wherein said cell is a brain cell.

39. The method of claim 36 or 37 wherein said β-amyloid is β-amyloid-42.

40. A method of treatment comprising modulating the production and/or release of β-amyloid in a human in need of said treatment, said method comprising administering to said human a compound according to any one of claims 1-15, 17 or 19-34, or a composition according to any one of claims 16, 18 and 35.

41. A method of treatment comprising modulating the production and/or release of β-amyloid in a human in need of said treatment, said method comprising administering to said human a compound of the formula 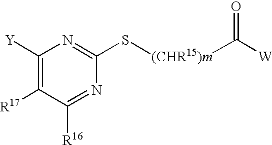
wherein, independently at each occurrence,
R¹⁵ and R¹⁷ are each independently selected from the group consisting of hydrogen and lower alkyl radicals;
R¹⁶ is selected from the group consisting of hydrogen, halogen and lower alkoxy radicals;
W is selected from the group consisting of hydroxy, lower alkoxy, —OM and —(NH)ₚNH₂ radicals, wherein p is 0 or 1, and M is an alkali metal cation, an alkaline earth metal cation or the ammonium ion;
m is 0, 1, 2 or 3;
Y is selected from the group consisting of an aryl radical of 6 to 10 carbon atoms; 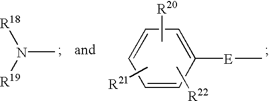
wherein
R¹⁸ is hydrogen or lower alkyl radical;
R¹⁹ is hydrogen, H₂N—, 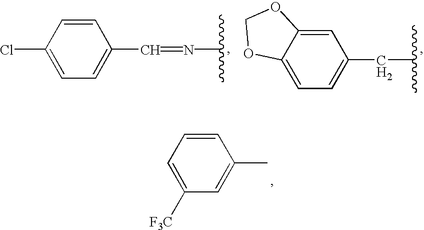
phenyl, (lower)alkoxyphenyl, or di(lower)alkoxy-phenyl, providing that when R¹⁸ is hydrogen and R¹⁹ is hydrogen, phenyl, (lower)alkoxyphenyl or di(lower)alkoxyphenyl, R¹⁶ is halo or lower alkoxy,
R²⁰ is selected from the group consisting of a lower alkyl radical, a halo radical, an aryl radical of 6 to 10 carbon atoms and a haloaryl radical of 6 to 10 carbon atoms,
R²¹ is selected from the group consisting of hydrogen, lower alkyl, lower alkoxy and halo radicals,
R²² is selected from the group consisting of hydrogen and lower alkyl radicals, and
E is selected from the group consisting of 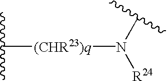
and 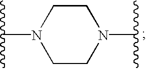
wherein
R²³ is hydrogen or lower alkyl,
R²⁴ is hydrogen or lower alkyl, and
q is an integer from 0 to 3.

42. A method of treatment comprising modulating the production and/or release of β-amyloid in a human in need of said treatment, said method comprising administering to said human a compound of the formula 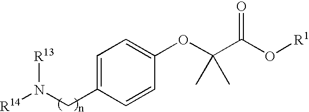
wherein,
R¹ is selected from the group consisting of C₁-C₃ alkyl, hydrogen, metal cation and ammonium cation;
R¹³ and R¹⁴ are each independently selected from the group consisting of hydrogen, alkyl, alkenyl, aryl, aralkyl, aralkenyl, cycloalkyl, cycloalkylalkyl, cycloalkylalkenyl, halogen, haloalkyl, haloalkenyl, cyano, nitro, —R¹⁰—N═N—O—R¹¹, —OR¹², —C(O)OR¹², —N(R¹²)₂, —C(O)N(R¹²)₂, —N(R¹²)C(O)OR¹¹, heterocyclyl and heterocyclylalkyl;
R¹⁰ is a bond or a straight or branched alkylene or alkenylene chain;
R¹¹ is hydrogen, alkyl or aralkyl; and
R¹² is independently selected from the group consisting of hydrogen, alkyl, alkenyl, haloalkyl, haloalkenyl, aryl, aralkyl, aralkenyl, cycloalkyl, cycloalkylalkyl and cycloalkylalkenyl; and
n is 1, 2 or 3.

43. The method of claims 40-42 wherein said human is afflicted with Alzheimer s disease.

44. The method of claims 40-42 wherein said human has suffered a head injury.

45. The method of claims 40-42 wherein said human has a genetic predisposition or environment exposure that increases the likelihood that said person will develop Alzheimer's disease.

46. The method of claims 40-42 wherein said human exhibits minimal cognitive impairment suggestive of early stage Alzheimer's disease.

47. A method of treatment comprising modulating the production and/or release of β-amyloid in a non-human mammal in need of said treatment, said method comprising administering to said non-human mammal a compound according to any one of claims 1-15, 17 or 19-34, or a composition according to any one of claims 16, 18 and 35.

48. A method of treatment comprising modulating the production and/or release of β-amyloid in a non-human mammal in need of said treatment, said method comprising administering to said non-human mammal a compound of the formula 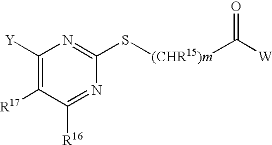
wherein, independently at each occurrence,
R¹⁵ and R¹⁷ are each independently selected from the group consisting of hydrogen and lower alkyl radicals;
R¹⁶ is selected from the group consisting of hydrogen, halogen and lower alkoxy radicals;
W is selected from the group consisting of hydroxy, lower alkoxy, —OM and —(NH)ₚNH₂ radicals, wherein p is 0 or 1, and M is an alkali metal cation, an alkaline earth metal cation or the ammonium ion;
m is 0, 1, 2 or 3;
Y is selected from the group consisting of an aryl radical of 6 to 10 carbon atoms; 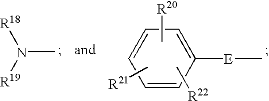
wherein
R¹⁸ is hydrogen or lower alkyl radical;
R¹⁹ is hydrogen, H₂N—, 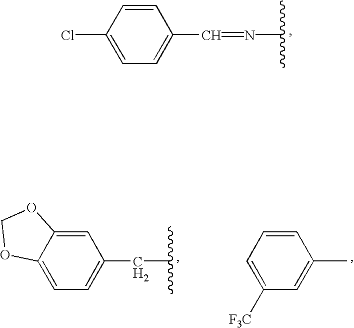
phenyl, (lower)alkoxyphenyl, or di(lower)alkoxy-phenyl, providing that when R¹⁸ is hydrogen and R¹⁹ is hydrogen, phenyl, (lower)alkoxyphenyl or di(lower)alkoxyphenyl, R¹⁶ is halo or lower alkoxy,
R²⁰ is selected from the group consisting of a lower alkyl radical, a halo radical, an aryl radical of 6 to 10 carbon atoms and a haloaryl radical of 6 to 10 carbon atoms,
R²¹ is selected from the group consisting of hydrogen, lower alkyl, lower alkoxy and halo radicals,
R²² is selected from the group consisting of hydrogen and lower alkyl radicals, and
E is selected from the group consisting of 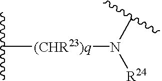
and 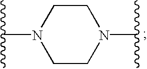
wherein
R²³ is hydrogen or lower alkyl,
R²⁴ is hydrogen or lower alkyl, and
q is an integer from 0 to 3.
